(19) United States
(12) Patent Application Publication
(10) Pub. No.: US 2006/0040853 A1
(43) Pub. Date: Feb. 23, 2006

(54) Methods and compositions useful for modulation of angiogenesis using protein kinase Raf and Ras

(75) Inventors: John Hood, Solana Beach, CA (US); Brian Eliceiri, Carlsbad, CA (US); David A. Cheresh, Encinitas, CA (US)

Correspondence Address: OLSON & HIERL, LTD., 20 NORTH WACKER DRIVE, 36TH FLOOR, CHICAGO, IL, 60606, US

(73) Assignee: The Scripps Research Institute

(21) Appl. No.: 11/219,264

(22) Filed: Sep. 2, 2005

Related U.S. Application Data
(62) Division of application No. 09/637,302, filed on Aug. 11, 2000, now abandoned.
(60) Provisional application No. 60/215,951, filed on Jul. 5, 2000.
(60) Provisional application No. 60/148,924, filed on Aug. 13, 1999.

(51) Int. Cl. A61K 38/48; A61K 9/127

(52) U.S. Cl. 514/2; 424/450

(57) ABSTRACT

The present invention describes methods for modulating angiogenesis in tissues using Raf and/or Ras protein, modified Raf or Ras protein, and nucleic acids encoding for such. Particularly the invention describes methods for inhibiting angiogenesis using an inactive Raf and/or Ras protein, or nucleic acids encoding therefor, or for potentiating angiogenesis using an active Raf and/or Ras protein, or nucleic acids encoding therefor. The invention also describes the use of gene delivery systems for providing nucleic acids encoding for the Raf or Ras protein, or modified forms thereof.

### CROSS-REFERENCE TO RELATED APPLICATIONS

This application is a division of co-pending U.S. patent application Ser. No. 09/637,302, filed on Aug. 11, 2000, which claims benefit of U.S. Provisional Patent Application No. 60/215,951 filed Jul. 5, 2000, and U.S. Provisional Patent Application No. 60/148,924, filed Aug. 13, 1999.

### GOVERNMENTAL RIGHTS

This invention was made with government support under Contract No. CA50286 by the National Institutes of Health. The government has certain rights in the invention.

### TECHNICAL FIELD

The present invention relates generally to the field of medicine, and relates specifically to methods and compositions for modulating angiogenesis of tissues using the protein kinase Raf or Ras, variants of Raf or Ras, using reagents which modulate Raf or Ras, and using nucleic acids encoding them.

### BACKGROUND

Angiogenesis is a process of tissue vascularization that involves the growth of new blood vessels into a tissue, and is also referred to as neo-vascularization. The process is mediated by the infiltration of endothelial cells and smooth muscle cells. The process is believed to proceed in any one of three ways: the vessels can sprout from pre-existing vessels, de-novo development of vessels can arise from precursor cells (vasculogenesis), or existing small vessels can enlarge in diameter. Blood et al., Bioch. Biophys. Acta, 1032:89-118 (1990).

Angiogenesis is an important process in neonatal growth, but is also important in wound healing and in the pathogenesis of a large variety of clinical diseases including tissue inflammation, arthritis, tumor growth, diabetic retinopathy, macular degeneration by neovascularization of the retina and like conditions. These clinical manifestations associated with angiogenesis are referred to as angiogenic diseases. Folkman et al., Science, 235:442-447 (1987). Angiogenesis is generally absent in adult or mature tissues, although it does occur in wound healing and in the corpus luteum growth cycle. See, for example, Moses et al., Science, 248:1408-1410 (1990).

It has been proposed that inhibition of angiogenesis would be a useful therapy for restricting tumor growth. Inhibition of angiogenesis has been proposed by (1) inhibition of release of “angiogenic molecules” such as bFGF (basic fibroblast growth factor), (2) neutralization of angiogenic molecules, such as by use of anti-bFGF antibodies, (3) use of inhibitors of vitronectin receptor αᵥβ₃, and (4) inhibition of endothelial cell response to angiogenic stimuli. This latter strategy has received attention, and Folkman et al., Cancer Biology, 3:89-96 (1992), have described several endothelial cell response inhibitors, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like that might be used to inhibit angiogenesis. For additional proposed inhibitors of angiogenesis, see Blood et al., Bioch. Biophys. Acta., 1032:89-118 (1990), Moses et al., Science, 248:1408-1410 (1990), Ingber et al., Lab. Invest., 59:44-51 (1988), and U.S. Pat. Nos. 5,092,885, 5,112,946, 5,192,744, 5,202,352, 5,753,230 and 5,766,591. None of the inhibitors of angiogenesis described in the foregoing references involve the Raf proteins, however.

For angiogenesis to occur, endothelial cells must first degrade and cross the blood vessel basement membrane in a manner similar to that used by tumor cells during invasion and metastasis formation.

It has been previously reported that angiogenesis depends on the interaction between vascular integrins and extracellular matrix proteins. Brooks et al., Science, 264:569-571 (1994). Furthermore, it was reported that programmed cell death (apoptosis) of angiogenic vascular cells is initiated by the interaction, which would be inhibited by certain antagonists of the vascular integrin αᵥβ₃. Brooks et al., Cell, 79:1157-1164 (1994). More recently, it has been reported that the binding of matrix metalloproteinase-2 (MMP-2) to vitronectin receptor (αᵥβ₅) can be inhibited using αᵥβ₅ antagonists, and thereby inhibit the enzymatic function of the proteinase. Brooks et al., Cell, 85:683-693 (1996).

### SUMMARY OF THE INVENTION

The present invention contemplates modulation of angiogenesis in tissues where that angiogenesis depends upon the activity of protein kinase Raf, also referred to generically herein as Raf.

Compositions and methods for modulating angiogenesis in a tissue associated with a disease condition are contemplated. A composition comprising an angiogenesis-modulating amount of a Raf protein is administered to tissue to be treated for a disease condition that responds to modulation of angiogenesis. The composition providing the Raf protein can contain purified protein, biologically active protein fragments, recombinantly produced Raf protein or protein fragments or fusion proteins, or gene/nucleic acid expression vectors for expressing a Raf protein.

Where the Raf protein is inactivated or inhibited, the modulation is an inhibition of angiogenesis. Where the Raf protein is active or activated, the modulation is a potentiation of angiogenesis.

The tissue to be treated can be any tissue in which modulation of angiogenesis is desirable. For angiogenesis inhibition, it is useful to treat diseased tissue where deleterious neovascularization is occurring. Exemplary tissues include inflamed tissue, solid tumors, metastases, tissues undergoing restenosis, and the like tissues.

For potentiation, it is useful to treat patients with hypoxic tissues such as those following stroke, myocardial infarction or associated with chronic ulcers, tissues in patients with ischemic limbs in which there is abnormal, i.e., poor circulation, due to diabetic or other conditions. Patients with chronic wounds that do not heal, and therefore could benefit from the increase in vascular cell proliferation and neovascularization, can be treated as well.

Particularly preferred is the use of Raf protein containing a modified amino acid sequence as described herein. Several particularly useful modified Raf proteins, including Raf fusion proteins such as Raf-caax and nucleic acid constructs which encode for the expression thereof are described herein and are within the purview of the present invention.

The present invention also encompasses a pharmaceutical composition suitable for inhibiting angiogenesis in a target mammalian tissue comprising a viral or non-viral gene transfer vector containing a nucleic acid, the nucleic acid having a nucleic acid segment encoding for a Raf protein, and the Raf protein having any amino acid residue at codon 375 except for lysine, and a pharmaceutically acceptable carrier or excipient. A particularly preferred embodiment utilizes Raf protein designated as Raf K375M and described in the examples below. Another inactive Raf construct is a nucleic acid which encodes for a Raf protein having the carboxy terminal portion deleted. One preferred embodiment utilizes a Raf protein designated Raf 1-305, which is an inactive Raf protein.

Also envisioned is a pharmaceutical composition suitable for stimulating angiogenesis in a target mammalian tissue and comprising a viral or non-viral gene transfer vector containing a nucleic acid having a segment encoding for a Raf protein having kinase activity and a pharmaceutically acceptable carrier or excipient therefor. A preferred nucleic acid encodes for an inhibitory Raf fusion protein that is Raf-caax. Another inhibitory Raf construct contains a nucleic acid encoding for a Raf protien having the amino terminal portion of the protein deleted. One preferred embodiment utilizes a Raf protein designated Raf 306-648, and described in the examples below.

The invention further contemplates modulation of angiogenesis in tissues by small GTPase Ras, also referred to generically herein as Ras, due to its role in signaling Raf, as described herein. Also envisioned is the modulation of angiogenesis in tissues utilizing the combination of Ras and Raf modulation. Such combined modulation can take the form of a single administration of combined formulations of protein, or nucleic acid encoding modulating protein, or the separate administration of individual doses, in an angiogenesis-modulating amout.

Compositions and methods for modulating angiogenesis in a tissue, associated with a disease condition are contemplated, where the modulation is directed to the Raf-mediated angiogenesis pathway via the Ras protein. A composition comprising an angiogenesis-modulating amount of a Ras protein is administered to tissue to be treated for a disease condition that responds to modulation of angiogenesis. The composition providing the Ras protein can contain purified protein, biologically active Ras protein fragments, recombinantly produced Ras protein or protein fragments or fusion proteins, or gene/nucleic acid expression vectors for expressing a Ras protein.

Where the Ras protein is inactivated or inhibited, the modulation is an inhibition of angiogenesis. Where the Ras protein is active or activated, the modulation is a potentiation of angiogenesis. Pharmaceutical compositions and methods of use for dominant negative Ras proteins, such as S17N Ras or V12C40 Ras, are contemplated for use in a manner similar to that for proteins of the Raf family. In a further aspect of this invention, pharmaceutical compositions and methods of use for dominant active Ras proteins, such as G12V Ras or V12S35 Ras, are contemplated for uses comparable to those for the Raf family proteins.

Further contemplated are methods for modulating angiogenesis in a tissue associated with a disease condition comprising administering an angiogenesis modulating amount of a pharmaceutical composition comprising a Raf protein or a nucleotide sequence capable of expressing Raf protein, and a Ras protein or a nucleotide sequence capable of expressing Ras protein. In such methods, where the desired modulation is an inhibition of angiogenesis, at least one or both of the Raf or Ras proteins is inactive. Where the desired modulation is a stimulation of angiogenesis, at least one or both of the Raf or Ras proteins are active.

### BRIEF DESCRIPTION OF THE DRAWINGS

In the drawings forming a portion of this disclosure:

FIGS. 1A-1D illustrate that ecotrophically packaged retrovirus only infects murine cells. Ecotrophic packaging cells were transfected with a retroviral construct encoding the b-Galactosidase (b-Gal) gene and the supernatant collected 24 hours later. Supernate containing the virus was placed on either murine-derived fibroblasts (FIG. 1A), murine-derived endothelial cells (FIG. 1B), human epithelial adenocarcinoma cells (FIG. 1C), or human melanoma cells (FIG. 1D) for 24 hours. b-Gal activity was visualized using standard methods.

FIG. 2 illustrates that bFGF-induced increases in Raf activity were blocked by prior infection with Raf K375M in a mouse endothelial cell line. Ecotrophic packaging cells were transfected with a retroviral construct encoding the defective Raf kinase gene and the supernatant collected 24 hours later. Supernate containing virus was placed on mouse endothelial cells for 24 hours. Cells were then treated with bFGF for 5 minutes and lysed. Raf kinase activity was quantified by the ability of immunoprecipitated Raf kinase to phosphorylate the MEK substrate with radioactively labeled ³²P. Reaction mixtures were fractionated by SDS PAGE and quantified using scanning densitometry.

FIGS. 3A-3B illustrate that mutant inactive Raf K375M blocks bFGF-induced angiogenesis in a murine subcutaneous angiogenesis model. Angiogenesis was induced by injecting 250 ul of ice-cold, growth factor-reduced matrigel containing 400 ng/ml bFGF, with or without retrovirus expressing packaging cells that express Raf K375M, subcutaneously in the mouse flank. Five days later endothelial-specific FITC-conjugated Bandeiriea Simplifica B5 lectin was injected via the tail vein and allowed to circulate and clear for 30 minutes. Angiogenesis was then quantitated by removing, extracting, and assaying the angiogenic tissue for fluorescent content (FIG. 3A). Neovascularization was confirmed by optical sectioning (FIG. 3B).

FIGS. 4A-4B illustrate that mutationally active Raf stimulates angiogenesis in a murine subcutaneous angiogenesis model. Angiogenesis was induced by injecting 250 ul of ice-cold, growth factor-reduced matrigel containing retrovirus expressing packaging cells which express GFP control or amino terminal deleted Raf kinase (Raf 306-648), subcutaneously in the mouse flank. Five days later angiogenesis was then quantitated by removing, extracting, and assaying the angiogenic tissue for fluorescent content (FIG. 4A). Neovascularization was confirmed by sectioning and staining with Mason's trichrome (FIG. 4B).

FIGS. 5A-5D illustrate retroviral delivery of Raf K375M kinase to the tumor induced apoptosis in an endothelial-specific manner. Human tumors were injected subcutaneously on the flank of athymic wehi (nu/nu) mice and allowed to implant. When tumors reached 100 mm³ they were injected intratumorally with culture supernate containing 10⁶ pfu of ecotrophically packaged Raf K375M. Forty-eight hours later the tumor was harvested, sectioned, and immunohistochemistry performed. Endothelial cells were identified by vWF expression (FIG. 5A), while the Flag tag marker was used to indicate cells infected by the Raf K375M kinase gene (FIG. 5B). Each of these markers are seen colocalized with the TUNEL marker indicative of apoptotic cells (FIGS. 5C & 5D).

FIGS. 6A-6B illustrate endothelial delivery of the Raf K375M kinase gene inhibited tumor growth and stimulated tumor regression. Human tumors were injected subcutaneously on the flank of athymic wehi (nu/nu) mice and allowed to grow to 100 mm³. At this point either a single injection of packaging cells expressing Raf K375M kinase was performed at a tumor-adjacent site or a series of intratumoral injections of viral supernate was initiated. This strategy resulted in rapid regressions of the tumors which was not seen with injection of the control GFP gene (FIG. 6A). This regression occurred rapidly and was maintained throughout the length of the experiment (FIG. 6B).

FIG. 7 depicts a cDNA sequence encoding for human c-Raf which is the complete coding sequence with the introns deleted. The sequence is accessible through GenBank Accession Number X03484 (GI=35841, HSRAFR). (SEQ ID NO.: 1).

FIG. 8 depicts the encoded translated amino acid residue sequence of human c-Raf of the coding sequence depicted in the nucleic acid sequence shown in FIG. 7. (SEQ ID NO.: 2).

FIG. 9 illustrates that angiogenesis is dependent on activation of the Ras-Raf-MEK-ERK pathway. Ras activity was elevated in chick chorioallantoic membrane (CAM) lysates exposed to bFGF as determined by a Ras pulldown assay. CAMs from 10-day old chick embryos were stimulated topically with filter disks saturated with either PBS or 30 nanograms (ng) of bFGF. After 5 minutes, CAM tissue was resected, homogenized in lysis buffer, and Ras activity was then determined by its capacity to be precipitated by a GST fusion peptide encoding the Ras binding domain of Raf. Because only active Ras binds Raf, a recombinant protein was generated consisting of the Ras binding domain of Raf conjugated to glutathione-S-transferase (GST). In turn GST was conjugated to sepharose beads enabling the precipitation of active Ras from a tissue lysate.

FIG. 10 depicts the cDNA coding domain nucleotide sequence of wild-type human Ras (wt H-Ras). (SEQ ID NO.: 3). A complete coding sequence for c-Ha-Ras1 proto-oncogene is accessible through GenBank (GI=190890, HUMRASH). (SEQ ID NO.: 5).

FIG. 11 depicts the amino acid residue sequence encoded by the cDNA nucleotide sequence of wild-type human Ras (wt H-Ras) shown in FIG. 10. (SEQ ID NO.: 4).

FIG. 12 illustrates that infection with mutant null Ras blocked growth factor-induced angiogenesis in the CAM. Fifteen microliters (ul) of high titer Chicken sarcoma retrovirus, RCAS(A), encoding mutant null Ras, S17N Ras (wild type H-Ras with a substitution of Asn for Ser at position 17), was topically applied to filter disks on CAMs as stimulated with bFGF as described in FIG. 9. Angiogenesis was assessed after 72 hours by counting vessel branch points.

FIGS. 13A and 13B illustrate schematically and graphically respectively that infection with a mutant Ras construct, Ras V12S35, which selectively activates the Ras-Raf-MEK-ERK pathway, induced angiogenesis, whereas a mutant construct, Ras V12C40, which selectively activates the P13K pathways, did not. Fifteen ul of high titer RCAS (A) virus encoding the Raf-MEK-ERK activating Ras construct, Ras V12S35, or the PI3 kinase activating Ras construct, Ras V12C40, were topically applied to filter disks and results assessed as described in FIG. 12.

FIG. 14 depicts the nucleotide sequence encoding the fusion protein Raf-caax, where the nucleotide sequence encoding the carboxy terminus of human Raf (wt H-Raf) is fused with a nucleotide sequence of encoding a 20 amino acid residue sequence of the K-Ras membrane localization domain. (SEQ ID NO.: 6).

FIG. 15 depicts the amino acid residue sequence of Raf-caax, the fusion protein generated from the fusion nucleotide sequence depicted in FIG. 14. (SEQ ID NO.: 7).

FIGS. 16A-16E and FIG. 16F, respectively, pictorially and graphically illustrate that the MEK inhibitor, PD98059, blocked angiogenesis induced by either mutant active Ras or Raf. Virus encoding the activating Ras construct, Ras V12 (also referred to as G12V, and the activating Raf construct, Raf-caax, were topically applied to filter disks as described in FIG. 12. After 24 hours, one (1) nanomole of the MEK inhibitor, PD98059, was added to the disk. The CAMs were then evaluated as described in FIG. 12. Data plotted is the mean±SE of 20 embryos.

FIGS. 17A-17F and FIG. 17G, respectively, pictorially and graphically illustrate that angiogenesis induced by Raf, but not Ras, was refractory to inhibition by integrin blockade. Infection with both mutant active Ras and Raf constructs induced pronounced angiogenesis, but only Ras-induced angiogenesis was inhibited by αᵥβ₃ integrin-blocking antibodies. CAMs from 10-day old chick embryos were stimulated as described in FIGS. 9 and 12 with filter disks saturated with either PBS (control), bFGF, the RCAS(A) retroviral constructs G12V-Ras or Raf-caax. LM609, a monoclonal antibody to integrin αᵥβ₃, was intravenously delivered after 24 hours and angiogenesis was assessed by vessel branch point analysis after 72 hours. Representative CAMs are shown in the inset. Data is the mean±SE of 20 embryos.

FIGS. 18A-18D and 18E, respectively, pictorially and graphically illustrate that co-infection of CAMs with a mutant null focal adhesion kinase, FRNK, blocked Ras, but not Raf-induced angiogenesis. RCAS(A) viruses encoding Ras V12 or Raf-caax were topically applied as described in FIG. 12 along with RCAS(B) virus encoding FAK-related-null-kinase (FRNK) to the CAM filter disk. Data is the mean±SE of 20 embryos.

FIGS. 19A and 19B-19G, respectively, graphically and pictorially, illustrate that FRNK blocked bFGF and Ras-, but not Raf, -induced angiogenesis in a murine subcutaneous angiogenesis model. Angiogenesis was induced by injecting 250 ul of ice-cold, growth factor-reduced matrigel containing either 400 ng/ml bFGF or Moloney retrovirus expressing packaging cells expressing the described gene, subcutaneously in the mouse flank. FRNK retrovirus was added to matrigel as high titer virus packaged with the vsv.g coat protein. Five days later, endothelial-specific FITC-conjugated Bandeiriea Simplifica B5 lectin was injected via the tail vein and allowed to circulate. Angiogenesis was then quantitated by removing, extracting, and assaying the angiogenic tissue for fluorescent content.

FIGS. 20A and 20B illustrate that co-infection of CAMs with a mutant null focal adhesion kinase, FRNK, blocked Ras-induced activation of Raf. CAMs were treated as described in FIG. 18 with the exception that after 24 hours the angiogenic tissue was resected, solubilized, Raf immunoprecipitated, and Raf activity assessed by its capacity to phosphorylate kinase-dead MEK. FIG. 20A shows the immunoprecipated active versus total Raf proteins assayed under each of the combinations above the results. FIG. 20B graphically plots the results of the active Raf determinations under those conditions.

### DETAILED DESCRIPTION OF THE INVENTION

A. Definitions

Amino Acid Residue: An amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxy terminus of a polypeptide. In keeping with standard polypeptide nomenclature (described in J. Biol. Chem., 243:3552-59 (1969) and adopted at 37 CFR §1.822(b)(2)).

It should be noted that all amino acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino acid residues.

Polypeptide: refers to a linear series of amino acid residues connected to one another by peptide bonds between the alpha-amino group and carboxy group of contiguous amino acid residues.

Peptide: as used herein refers to a linear series of no more than about 50 amino acid residues connected one to the other as in a polypeptide.

Cyclic peptide: refers to a compound having a heteroatom ring structure that includes several amide bonds as in a typical peptide. The cyclic peptide can be a “head to tail” cyclized linear polypeptide in which a linear peptide's n-terminus has formed an amide bond with the terminal carboxylate of the linear peptide, or it can contain a ring structure in which the polymer is homodetic or heterodetic and comprises amide bonds and/or other bonds to close the ring, such as disulfide bridges, thioesters, thioamides, guanidino, and the like linkages.

Protein: refers to a linear series of greater than 50 amino acid residues connected one to the other as in a polypeptide.

Fusion protein: refers to a polypeptide containing at least two different polypeptide domains operatively linked by a typical peptide bond (“fused”), where the two domains correspond to peptides not found fused in nature.

Synthetic peptide: refers to a chemically produced chain of amino acid residues linked together by peptide bonds that is free of naturally occurring proteins and fragments thereof.

B. General Considerations

The present invention relates generally to the discovery that angiogenesis is mediated by the protein kinase Raf protein, and that angiogenesis can be modulated by providing either active or inactive Raf proteins for potentiating or inhibiting angiogenesis, respectively. The invention also relates to the discovery that a Ras protein can affect Raf, and thereby modulate angiogenesis.

This discovery is important because of the role that angiogenesis, the formation of new blood vessels, plays in a variety of disease processes. On the other hand, where tissues associated with a disease condition require angiogenesis for tissue growth, it is desirable to inhibit angiogenesis and thereby inhibit the diseased tissue growth. Where injured tissue requires angiogenesis for tissue growth and healing, it is desirable to potentiate or promote angiogenesis and thereby promote tissue healing and growth.

Where the growth of new blood vessels is the cause of, or contributes to, the pathology associated with a disease tissue, inhibition of angiogenesis will reduce the deleterious effects of the disease. By inhibiting angiogenesis, one can intervene in the disease, ameliorate the symptoms, and in some cases cure the disease.

Examples of tissue associated with disease and neovascularization that will benefit from inhibitory modulation of angiogenesis include cancer, rheumatoid arthritis, ocular diseases such as diabetic retinopathy, inflammatory diseases, restenosis, and the like. Where the growth of new blood vessels is required to support growth of a deleterious tissue, inhibition of angiogenesis reduces the blood supply to the tissue and thereby contributes to reduction in tissue mass based on blood supply requirements. Particularly preferred examples include growth of tumors where neovascularization is a continual requirement in order that the tumor grow beyond a few millimeters in thickness, and for the establishment of solid tumor metastases.

Where the growth of new blood vessels contributes to healing of tissue, potentiation of angiogenesis assists in healing. Examples include treatment of patients with ischemic limbs in which there is abnormal, i.e. poor circulation as a result of diabetes or other conditions. Also contemplated are patients with chronic wounds which do not heal and therefore could benefit from the increase in vascular cell proliferation and neovascularization.

The methods of the present invention are effective in part because the therapy is highly selective for angiogenesis and not other biological processes.

As described earlier, angiogenesis includes a variety of processes involving neovascularization of a tissue including “sprouting”, vasculogenesis, or vessel enlargement, all of which angiogenesis processes are affected by Raf protein alone or together with a Ras protein. With the exception of traumatic wound healing, corpus luteum formation and embryogenesis, it is believed that the majority of angiogenesis processes are associated with disease processes and therefore the use of the present therapeutic methods are selective for the disease and do not have deleterious side effects.

C. Raf Proteins

A protein kinase Raf protein for use in the present invention can vary depending upon the intended use. The terms “Raf protein” or “Raf” are used to refer collectively to the various forms of protein kinase Raf protein, either in active or inactive forms.

An “active Raf protein” refers to any of a variety of forms of Raf protein which potentiate, stimulate, activate, induce or increase angiogenesis. Assays to measure potentiation of angiogenesis are described herein, and are not to be construed as limiting. A protein is considered active if the level of angiogenesis is at least 10% greater, preferably 25% greater, and more preferably 50% greater than a control level where no Raf is added to the assay system. The preferred assay for measuring potentiation is the in vitro Raf kinase as described in the Examples in which MEK substrate is phosphorylated with ³²P. Exemplary active Raf proteins are described in the Examples.

An “inactive Raf protein” refers to any of a variety of forms of Raf protein which inhibit, reduce, impede, or restrict angiogenesis. Assays to measure inhibition of angiogenesis are described herein, and are not to be construed as limiting. A protein is considered inactive if the level of angiogenesis is at least 10% lower, preferably 25% lower, and more preferably 50% lower than a control level where no exogenous Raf is added to the assay system. The preferred assay for measuring inhibition is the in vitro Raf kinase as described in the Examples in which MEK substrate is phosphorylated with ³²P. Exemplary inactive Raf proteins are described in the Examples.

A Raf protein useful in the present invention can be produced in any of a variety of methods including isolation from natural sources including tissue, production by recombinant DNA expression and purification, and the like. Raf protein can also be provided “in situ” by introduction of a gene therapy system to the tissue of interest which then expresses the protein in the tissue.

A gene encoding a Raf protein can be prepared by a variety of methods known in the art, and the invention is not to be construed as limiting in this regard. For example, the natural history of Raf is well known to include a variety of homologs from mammalian, avian, viral and the like species, and the gene can readily be cloned using cDNA cloning methods from any tissue expressing the protein. A preferred Raf for use in the invention is a cellular protein, such as the mammalian or avian homologs designated c-Raf. Particularly preferred is human c-Raf. A further preferred Raf protein of this invention is a fusion protein of Raf that is constitutively active but independent of Ras-mediated activation. Such a Raf protein can be a fusion protein. A preferred Ras-independent Raf protein is Raf-caax which is a carboxy terminal fusion protein of wild type Raf with the K-Ras membrane localization domain as further described in the Examples.

D. Ras Proteins

Ras family GTPases for use in the present invention can vary depending upon the intended use. The terms “Ras protein” or “Ras” are used herein to refer collectively to the various forms of Ras protein, either in active or inactive forms.

An “active Ras protein” refers to any of a variety of forms of Ras protein which potentiate, stimulate, activate, induce or increase angiogenesis. Assays to measure potentiation of angiogenesis by Ras are described herein, and are not to be construed as limiting. A protein is considered active if the level of angiogenesis is at least 10% greater, preferably 25% greater, and more preferably 50% greater than a control level where no Ras is added to the assay system. Exemplary active Ras proteins are Ras G12V, also referred to as V12, and Ras V12S35, both of which are further described in the Examples.

An “inactive Ras protein” refers to any of a variety of forms of Ras protein which inhibit, impede, delay, or stop angiogenesis. Assays to measure inhibition of angiogenesis are described herein, and are not to be construed as limiting. A protein is considered inactive if the level of angiogenesis is at least 10% lower, preferably 25% lower, and more preferably 50% lower than a control level where no exogenous Ras is added to the assay system. Exemplary inactive Ras proteins include the null mutant Ras referred to as Ras S17N (or sometimes N17) and V12C40, both of which are further described in the Examples.

A Ras protein useful in the present invention can be produced in any of a variety of methods including isolation from natural sources including tissue, production by recombinant DNA expression and purification, and the like. Ras protein can also be provided “in situ” by introduction of a gene therapy system to the tissue of interest which then expresses the protein in the tissue.

A gene encoding a Ras protein can be prepared by a variety of methods known in the art. The present invention is not to be construed as limiting in this regard. For example, the natural history of Ras is well known to include a variety of homologs from mammalian, avian, viral and the like species, and the gene can readily be cloned using cDNA cloning methods from any tissue expressing the protein.

It is to be understood by the present teachings that a Ras protein in its collective forms can be used in the same various embodiments as is described herein for a Raf protein, and therefore, the details for using a Ras protein are not reiterated. For example, Ras may be presented in an active or inactive form for modulating angiogenesis, or may be provided by nucleic acid expression of the Ras protein product, through the use of vector delivery systems, and in various pharmaceutical (therapeutic) compositions and articles of manufacture for practicing the invention. Methods of modulating angiogenesis using a Ras-based reagent in place of the recited Raf-based reagents are also contemplated.

E. Recombinant DNA Molecules and Expression Systems for Expression of a Raf or Ras Protein

The invention describes several nucleotide sequences of particular use in the present invention. These define nucleic acid sequences which encode for Raf or Ras protein useful in the invention, and various DNA segments, recombinant DNA (rDNA) molecules and vectors constructed for expression of Raf and/or Ras protein.

DNA molecules (segments) of this invention therefore can comprise sequences which encode whole structural genes, fragments of structural genes, and transcription units as described further herein.

A preferred DNA segment is a nucleotide sequence which encodes a Raf protein as defined herein, or biologically active fragment thereof.

Another preferred DNA segment is a nucleotide sequence which encodes a Ras protein as defined herein, or biologically active fragment thereof. By biologically active, it is meant that the expressed protein will have at least some of the biological activity of the intact protein found in a cell, such as ligand binding, or in the case of active forms of the protein, enzymatic activity.

The amino acid residue sequence and nucleotide sequence of a preferred c-Raf and h-Ras are described in the Examples.

A preferred DNA segment codes for an amino acid residue sequence substantially the same as, and preferably consisting essentially of, an amino acid residue sequence or portions thereof corresponding to a Raf or Ras protein described herein. Representative and preferred DNA segments are further described in the Examples.

The amino acid residue sequence of a protein or polypeptide is directly related via the genetic code to the deoxyribonucleic acid (DNA) sequence of the structural gene that codes for the protein. Thus, a structural gene or DNA segment can be defined in terms of the amino acid residue sequence, i.e., protein or polypeptide, for which it codes.

An important and well known feature of the genetic code is its redundancy. That is, for most of the amino acids used to make proteins, more than one coding nucleotide triplet (codon) can code for or designate a particular amino acid residue. Therefore, a number of different nucleotide sequences may code for a particular amino acid residue sequence. Such nucleotide sequences are functionally equivalent since they can result in the production of the same amino acid residue sequence in all organisms. Occasionally, a methylated variant of a purine or pyrimidine may be incorporated into a given nucleotide sequence. However, such methylations do not affect the coding relationship in any way.

A nucleic acid is any polynucleotide or nucleic acid fragment, whether it be a polyribonucleotide of polydeoxyribonucleotide, i.e., RNA or DNA, or analogs thereof. In preferred embodiments, a nucleic acid molecule is in the form of a segment of duplex DNA, i.e, a DNA segment, although for certain molecular biological methodologies, single-stranded DNA or RNA is preferred.

DNA segments are produced by a number of means including chemical synthesis methods and recombinant approaches, preferably by cloning or by polymerase chain reaction (PCR). DNA segments that encode all or only portions of a Raf or Ras protein can easily be synthesized by chemical techniques, for example, the phosphotriester method of Matteucci et al, J. Am. Chem. Soc., 103:3185-3191 (1981), or using automated synthesis methods. In addition, larger DNA segments can readily be prepared by well known methods, such as synthesis of a group of oligonucleotides that define the DNA segment, followed by hybridization and ligation of oligonucleotides to build the complete segment. Alternative methods include isolation of a preferred DNA segment by PCR with a pair of oligonucleotide primers used on a cDNA library believed to contain members which encode a Raf or Ras protein.

Of course, through chemical synthesis, any desired modifications can be made simply by substituting the appropriate bases for those encoding the native amino acid residue sequence. This method is well known, and can be readily applied to the production of the various different “modified” Raf or Ras proteins described herein.

Furthermore, DNA segments consisting essentially of structural genes encoding a Raf or Ras protein can be subsequently modified, as by site-directed or random mutagenesis, to introduce any desired substitutions. It is understood that various allelic forms of Raf or Ras protein and genes encoding for Raf or Ras protein are also suitable for use in the present invention.

1. Cloning a Raf or Ras Gene

A Raf or Ras gene of this invention can be cloned from a suitable source of genomic DNA or messenger RNA (mRNA) by a variety of biochemical methods. Cloning these genes can be conducted according to the general methods described in the Examples and as known in the art.

Sources of nucleic acids for cloning a Raf or Ras gene suitable for use in the methods of this invention can include genomic DNA or messenger RNA (mRNA) in the form of a cDNA library, from a tissue believed to express these proteins. A preferred tissue is human lung tissue, although any other suitable tissue may be used.

A preferred cloning method involves the preparation of a cDNA library using standard methods, and isolating the Raf-encoding or Ras-encoding nucleotide sequence by PCR amplification using paired oligonucleotide primers based on the nucleotide sequences described herein. Alternatively, the desired cDNA clones can be identified and isolated from a cDNA or genomic library by conventional nucleic acid hybridization methods using a hybridization probe based on the nucleic acid sequences described herein. Other methods of isolating and cloning suitable Raf-encoding or Ras-encoding nucleic acids are readily apparent to one skilled in the art.

2. Expression Vectors

The invention contemplates a recombinant DNA molecule (rDNA) containing a DNA segment encoding a Raf and/or Ras protein as described herein. An expressible rDNA can be produced by operatively (in frame, expressibly) linking a vector to a Raf or Ras encoding DNA segment of the present invention. It is envisioned that a combination expression can be constructed wherein Raf encoding and Ras encoding nucleic acid are present, either operably linked to the same, or separate promotors. Thus, a recombinant DNA molecule is a hybrid DNA molecule comprising at least two nucleic acids of a nucleotide sequences not normally found together in nature (i.e. gene and vector).

The choice of vector to which a DNA segment of the present invention is operatively linked depends directly, as is well known in the art, on the functional properties desired, e.g., protein expression, and the host cell to be transformed. Typical considerations in the art of constructing recombinant DNA molecules. A vector contemplated by the present invention is at least capable of directing the replication, and preferably also expression, of a structural gene included in the vector DNA segments, to which it is operatively linked.

Both prokaryotic and eukaryotic expression vectors are familiar to one of ordinary skill in the art of vector construction, and are described by Ausebel, et al., in Current Protocols in Molecular Biology, Wiley and Sons, New York (1993) and by Sambrook et al., Molecular Cloning: A Laboratory Manual, Cold Spring Harbor Laboratory (1989). These references also describe many of the general recombinant DNA methods referred to herein.

In one embodiment, a vector contemplated by the present invention includes a procaryotic replicon, i.e., a DNA sequence having the ability to direct autonomous replication and maintenance of the recombinant DNA molecule extrachromosomally in a procaryotic host cell, such as a bacterial host cell, transformed therewith. Such replicons are well known in the art. In addition, those embodiments that include a procaryotic replicon also include a gene whose expression confers drug resistance to a bacterial host transformed therewith. Typical bacterial drug resistance genes are those that confer resistance to ampicillin or tetracycline.

Those vectors that include a procaryotic replicon can also include a procaryotic promoter capable of directing the expression (transcription and translation) of a structural gene in a bacterial host cell, such as E. coli, transformed therewith. A promoter is an expression control element formed by a DNA sequence that permits binding of RNA polymerase and transcription to occur. Promoters or other such regulatory nucleic acid sequences can be inducible or constitutive depending upon the desired expression control and/or effect. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from Biorad Laboratories, (Richmond, Calif.), pRSET available from Invitrogen (San Diego, Calif.) and pPL and pKK223 available from Pharmacia, Piscataway, N.J.

Expression vectors compatible with eukaryotic cells, preferably those compatible with vertebrate cells, can also be used to form the recombinant DNA molecules of the present invention. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), pRc/CMV (Invitrogen, Inc.), the preferred vector described in the Examples, and the like eukaryotic expression vectors.

A particularly preferred system for gene expression in the context of this invention includes a gene delivery component, that is, the ability to deliver the gene to the tissue of interest. Suitable vectors are “infectious” vectors such as recombinant DNA viruses, adenovirus or retrovirus vectors which are engineered to express the desired protein and have features which allow infection of preselected target tissues. Particularly preferred is the retrovirus vector system described herein.

Mammalian cell systems that utilize recombinant viruses or viral elements to direct expression may be engineered. For example, when using adenovirus expression vectors, the coding sequence of a polypeptide may be ligated to an adenovirus transcription/translation control complex, e.g., the late promoter and tripartite leader sequence. This chimeric gene may then be inserted into the adenovirus genome by in vitro or in vivo recombination. Insertion in a non-essential region of the viral genome (e.g., region E1 or E3) will result in a recombinant virus that is viable and capable of expressing the polypeptide in infected hosts (e.g., see Logan et al., Proc. Natl. Acad. Sci., USA, 81:3655-3659 (1984)). Alternatively, the vaccinia virus 7.5K promoter may be used (e.g., see, Mackett et al., Proc. Natl. Acad. Sci., USA, 79:7415-7419 (1982); Mackett et al., J. Virol., 49:857-864 (1984); Panicali et al., Proc. Natl. Acad. Sci. USA, 79:4927-4931 (1982)). Of particular interest are vectors based on bovine papilloma virus which have the ability to replicate as extrachromosomal elements (Sarver et al., Mol. Cell. Biol., 1:486 (1981)). Shortly after entry of this DNA into target cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression of the polypeptide-encoding nucleotide sequence in host cells (Cone et al., Proc. Natl. Acad. Sci. USA, 81:6349-6353 (1984)). High level expression may also be achieved using inducible promoters, including, but not limited to, the metallothionine IIA promoter and heat shock promoters.

Recently, long-term survival of cytomegalovirus (CMV) promoter versus Rous sarcoma virus (RSV) promotor-driven thymidine kinase (TK) gene therapy in nude mice bearing human ovarian cancer has been studied. Cell killing efficacy of adenovirus-mediated CMV promoter-driven herpes simplex virus TK gene therapy was found to be 2 to 10 times more effective than RSV driven therapy (Tong et al., Hybridoma 18(1):93-97 (1999)). The design of chimeric promoters for gene therapy applications, which call for low level expression followed by inducible high-level expression has also been described (Suzuki et al., Human Gene Therapy 7:1883-1893 (1996)).

For long-term, high-yield production of recombinant proteins, stable expression is preferred. Rather than using expression vectors which contain viral origins of replication, host cells can be transformed with a cDNA controlled by appropriate expression control elements (e.g., promoter and enhancer sequences, transcription terminators, polyadenylation sites, etc.), and a selectable marker. As mentioned above, the selectable marker in the recombinant plasmid confers resistance to the selection and allows cells to stably integrate the plasmid into their chromosomes and grow to form foci which in turn can be cloned and expanded into cell lines.

For example, following the introduction of foreign DNA, engineered cells may be allowed to grow for 1-2 days in an enriched media, and then are switched to a selective media. A number of selection systems may be used, including but not limited to the herpes simplex virus thymidine kinase (Wigler et al., Cell, 11:223 (1977)), hypoxanthine-guanine phosphoribosyltransferase (Szybalska et al, Proc. Natl. Acad. Sci., USA, 48:2026 (1962)), and adenine phosphoribosyltransferase (Lowy et al., Cell, 22:817 (1980)) genes, which can be employed in tk⁻, hgprt⁻ or aprt⁻ cells respectively. Also, antimetabolite resistance-conferring genes can be used as the basis of selection; for example, the genes for dhfr, which confers resistance to methotrexate (Wigler et al., Proc. Natl. Acad. Sci., USA, 77:3567 (1980); O'Hare et al., Proc. Natl. Acad. Sci., USA, 78:1527 (1981); gpt, which confers resistance to mycophenolic acid (Mulligan et al, Proc. Natl. Acad. Sci., USA, 78:2072 (1981)); neo, which confers resistance to the aminoglycoside G-418 (Colberre-Garapin et al, J. Mol. Biol., 150:1 (1981)); and hygro, which confers resistance to hygromycin (Santerre et al, Gene, 30:147 (1984)). Recently, additional selectable genes have been described, namely trpB, which allows cells to utilize indole in place of tryptophan; hisD, which allows cells to utilize histinol in place of histidine (Hartman et al, Proc. Natl. Acad. Sci., USA, 85:804 (1988)); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory ed. (1987)).

The principal vectors contemplated for human gene therapy, are derived from retroviral origin (Wilson, Clin. Exp. Immunol. 107(Sup. 1):31-32 (1997); Bank et al., Bioessays 18(12):999-1007 (1996); Robbins et al., Pharmacol. Ther. 80(1):35-47 (1998)). The therapeutic potential of gene transfer and antisense therapy has stimulated the development of many vector systems for treating a variety of tissues (vasculature, Stephan et al., Fundam. Clin. Pharmacol. 11(2):97-110 (1997); Feldman et al., Cardiovasc. Res. 35(3):391-404 (1997); Vassalli et al., Cardiovasc. Res. 35(3):459-69 (1997); Baek et al., Circ. Res. 82(3):295-305 (1998); kidney, Lien et al., Kidney Int. Suppl. 61:S85-8 (1997); liver, Ferry et al., Hum Gene Ther. 9(14):1975-81 (1998); muscle, Marshall et al., Curr. Opn. Genet. Dev. 8(3):360-5 (1998)). In addition to these tissues, a critical target for human gene therapy is cancer, either the tumor itself, or associated tissues. (Runnebaum, Anticancer Res. 17(4B):2887-90 (1997); Spear et al., J. Neurovirol. 4(2): 133-47 (1998)).

Specific examples of viral gene therapy vector systems readily adaptable for use in the methods of the present invention are briefly described below. Retroviral gene delivery has been recently reviewed by Federspiel and Hughes (Methods in Cell Biol. 52:179-214 (1998)) which describes in particular, the avian leukosis virus (ALV) retrovirus family (Federspiel et al., Proc. Natl. Acad. Sci., USA, 93:4931 (1996); Federspiel et al., Proc. Natl. Acad. Sci., USA, 91:11241 (1994)). Retroviral vectors, including ALV and murine leukemia virus (MLV) are further described by Svoboda (Gene 206:153-163 (1998)).

Modified retroviral/adenoviral expression systems can be readily adapted for practice of the methods of the present invention. For example, murine leukemia virus (MLV) systems are reviewed by Karavanas et al., Crit. Rev. in Oncology/Hematology 28:7-30 (1998). Adenovirus expression systems are reviewed by Von Seggem and Nemerow in Gene Expression Systems (ed. Fernandez & Hoeffler, Academic Press, San Diego, Calif., chapter 5, pages 112-157 (1999)).

Protein expression systems have been demonstrated to have effective use both in vivo and in vitro. For example, efficient gene transfer to human squamous cell carcinomas by a herpes simplex virus (HSV) type 1 amplicon vector has been described. (Carew et al., 1998, Am. J. Surg. 176:404-408). Herpes simplex virus has been used for gene transfer to the nervous system (Goins et al., J. Neurovirol. 3 (Sup. 1):S80-8 (1997)). Targeted suicide vectors using HSV-TK has been tested on solid tumors (Smiley et al., Hum. Gene Ther. 8(8):965-77 (1997)). Herpes simplex virus type 1 vector has been used for cancer gene therapy on colon carcinoma cells (Yoon et al., Ann. Surg. 228(3):366-74 (1998)). Hybrid vectors have been developed to extend the length of time of transfection, including HSV/AAV (adeno-associated virus) hybrids for treating hepatocytes (Fraefel et al., Mol. Med. 3(12):813-825 (1997)).

Vaccinia virus has been developed for human gene therapy because of its large genome (Peplinski et al., Surg. Oncol. Clin. N. Am. 7(3):575-88 (1998)). Thymidine kinase-deleted vaccinia virus expressing purine nucleoside pyrophosphorylase has been described for use as a tumor directed gene therapy vector. (Puhlman et al., Human Gene Therapy 10:649-657 (1999)).

Adeno-associated virus 2 (AAV) has been described for use in human gene therapy, however AAV requires a helper virus (such as adenovirus or herpes virus) for optimal replication and packaging in mammalian cells (Snoeck et al., Exp. Nephrol. 5(6):514-20 (1997); Rabinowitz et al., Curr. Opn. Biotechnol. 9(5):470-5 (1998)). However, in vitro packaging of an infectious recombinant AAV has been described, making this system much more promising (Ding et al., Gene Therapy 4:1167-1172 (1997)). It has been shown that the AAV mediated transfer of ecotropic retrovirus receptor cDNA allows ecotropic retroviral transduction of established and primary human cells (Qing et al., J. Virology 71(7):5663-5667 (1997)). Cancer gene therapy using an AAV vector expressing human wild-type p53 has been demonstrated (Qazilbash et al., Gene Therapy 4:675-682 (1997)). Gene transfer into vascular cells using AAV vectors has also been shown (Maeda et al., Cardiovascular Res. 35:514-521 (1997)). AAV has been demonstrated as a suitable vector for liver directed gene therapy (Xiao et al., J. Virol. 72(12): 10222-6 (1998)). AAV vectors have been demonstrated for use in gene therapy of brain tissues and the central nervous system (Chamberlin et al., Brain Res. 793(1-2):169-75 (1998); During et al., Gene Therapy 5(6):820-7 (1998)). AAV vectors have also been compared with adenovirus vectors (AdV) for gene therapy of the lung and transfer to human cystic fibrosis epithelial cells (Teramoto et al., J. Virol. 72(11):8904-12 (1998)).

Chimeric AdV/retroviral gene therapy vector systems which incorporate the useful qualities of each virus to create a nonintegrative AdV that is rendered functionally integrative via the intermediate generation of a retroviral producer cell (Feng et al., Nat. Biotechnology 15(9):866-70 (1997); Bilbao et al., FASEB J 11(8):624-34 (1997)). This powerful new generation of gene therapy vector has been adapted for targeted cancer gene therapy (Bilbao et al., Adv. Exp. Med. Biol. 451:365-74 (1998)). Single injection of AdV expressing p53 inhibited growth of subcutaneous tumor nodules of human prostrate cancer cells (Asgari et al., Int. J. Cancer 71(3):377-82 (1997)). AdV mediated gene transfer of wild-type p53 in patients with advanced non-small cell lung cancer has been described (Schuler et al., Human Gene Therapy 9:2075-2082 (1998)). This same cancer has been the subject of p53 gene replacement therapy mediated by AdV vectors (Roth et al., Semin. Oncol. 25(3 Suppl 8):33-7 (1998)). AdV mediated gene transfer of p53 inhibits endothelial cell differentiation and angiogenesis in vivo (Riccioni et al., Gene Ther. 5(6):747-54 (1998)). Adenovirus-mediated expression of melanoma antigen gp75 as immunotherapy for metastatic melanoma has also been described (Hirschowitz et al., Gene Therapy 5:975-983 (1998)). AdV facilitates infection of human cells with ecotropic retrovirus and increases efficiency of retroviral infection (Scott-Taylor, et al., Gene Ther. 5(5):621-9 (1998)). AdV vectors have been used for gene transfer to vascular smooth muscle cells (Li et al., Chin. Med. J. (Engl) 110(12):950-4 (1997)), squamous cell carcinoma cells (Goebel et al., Otolarynol Head Neck Surg 119(4):331-6 (1998)), esophageal cancer cells (Senmaru et al., Int J. Cancer 78(3):366-71 (1998)), mesangial cells (Nahman et al., J. Investig. Med. 46(5):204-9 (1998)), glial cells (Chen et al., Cancer Res. 58(16):3504-7 (1998)), and to the joints of animals (Ikeda et al., J. Rheumatol. 25(9): 1666-73 (1998)). More recently, catheter-based pericardial gene transfer mediated by AcV vectors has been demonstrated (March et al., Clin. Cardiol. 22(1 Suppl 1):I23-9 (1999)). Manipulation of the AdV system with the proper controlling genetic elements allows for the AdV-mediated regulatable target gene expression in vivo (Burcin et al., PNAS (USA) 96(2):355-60 (1999)).

Alphavirus vectors have been developed for human gene therapy applications, with packaging cell lines suitable for transformation with expression cassettes suitable for use with Sindbis virus and Semliki Forest virus-derived vectors (Polo et al., Proc. Natl. Acad. Sci., USA, 96:4598-4603 (1999)). Noncytopathic flavivirus replicon RNA-based systems have also been developed (Varnavski et al., Virology 255(2):366-75 (1999)). Suicide HSV-TK gene containing sinbis virus vectors have been used for cell-specific targeting into tumor cells (Iijima et al., Int. J. Cancer 80(1): 110-8 (1998)).

Retroviral vectors based on human foamy virus (HFV) also show promise as gene therapy vectors (Trowbridge et al., Human Gene Therapy 9:2517-2525 (1998)). Foamy virus vectors have been designed for suicide gene therapy (Nestler et al., Gene Ther. 4(11): 1270-7 (1997)). Recombinant murine cytomegalovirus and promoter systems have also been used as vectors for high level expression (Manning et al., J. Virol. Meth. 73(1):31-9 (1998); Tong et al., Hebridoma 18(1):93-7 (1998)).

Gene delivery into non-dividing cells has been made feasible by the generation of Sendai virus based vectors (Nakanishi et al., J. Controlled Release 54(1):61-8 (1998)).

In other efforts to enable the transformation of non-dividing somatic cells, lentiviral vectors have been explored. Gene therapy of cystic fibrosis using a replication-defective human immunodeficiency virus (HIV) based vector has been described. (Goldman et al., Human Gene Therapy 8:2261-2268 (1997)). Sustained expression of genes delivered into liver and muscle by lentiviral vectors has also been shown (Kafri et al., Nat. Genet. 17(3):314-7 (1997)). However, safety concerns are predominant, and improved vector development is proceeding rapidly (Kim et al., J. Virol. 72(2):994-1004 (1998)). Examination of the HIV LTR and Tat yield important information about the organization of the genome for developing vectors (Sadaie et al., J. Med. Virol. 54(2):118-28 (1998)). Thus, the genetic requirements for an effective HIV based vector are now better understood (Gasmi et al., J. Virol. 73(3):1828-34 (1999)). Self inactivating vectors, or conditional packaging cell lines have been described (for example, Zuffery et al., J. Virol. 72(12):9873-80 (1998); Miyoshi et al., J. Virol. 72(10):8150-7 (1998); Dull et al., J. Virol. 72(11):8463-71 (1998); and Kaul et al., Virology 249(1):167-74 (1998)). Efficient transduction of human lymphocytes and CD34+cells by HIV vectors has been shown (Douglas et al., Hum. Gene Ther. 10(6):935-45 (1999); Miyoshi et al., Science 283(5402):682-6 (1999)). Efficient transduction of nondividing human cells by feline immunodeficiency virus (FIV) lentiviral vectors has been described, which minimizes safety concerns with using HIV based vectors (Poeschla et al., Nature Medicine 4(3):354-357 (1998)). Productive infection of human blood mononuclear cells by FUV vectors has been shown (Johnston et al., J. Virol. 73(3):2491-8 (1999)).

While many viral vectors are difficult to handle, and capacity for inserted DNA limited, these limitations and disadvantages have been addressed. For example, in addition to simplified viral packaging cell lines, Mini-viral vectors, derived from human herpes virus, herpes simplex virus type 1 (HSV-1), and Epstein-Barr virus (EBV), have been developed to simplify manipulation of genetic material and generation of viral vectors (Wang et al., J. Virology 70(12):8422-8430 (1996)). Adaptor plasmids have been previously shown to simplify insertion of foreign DNA into helper-independent Retroviral vectors (J. Virology 61(10):3004-3012 (1987)).

Viral vectors are not the only means for effecting gene therapy, as several non-viral vectors have also been described. A targeted non-viral gene delivery vector based on the use of Epidermal Growth Factor/DNA polyplex (EGF/DNA) has been shown to result in efficient and specific gene delivery (Cristiano, Anticancer Res. 18:3241-3246 (1998)). Gene therapy of the vasculature and CNS have been demonstrated using cationic liposomes (Yang et al., J. Neurotrauma 14(5):281-97 (1997)). Transient gene therapy of pancreatitis has also been accomplished using cationic liposomes (Denham et al., Ann. Surg. 227(6):812-20 (1998)). A chitosan-based vector/DNA complexes for gene delivery have been shown to be effective (Erbacher et al., Pharm. Res. 15(9): 1332-9 (1998)). A non-viral DNA delivery vector based on a terplex system has been described (Kim et al., 53(1-3):175-82 (1998)). Virus particle coated liposome complexes have also been used to effect gene transfer (Hirai et al., Biochem. Biophys. Res. Commun. 241(1):112-8 (1997)).

Cancer gene therapy by direct tumor injections of nonviral T7 vector encoding a thymidine kinase gene has been demonstrated (Chen et al., Human Gene Therapy 9:729-736 (1998)). Plasmid DNA preparation is important for direct injection gene transfer (Horn et al., Hum. Gene Ther. 6(5):656-73 (1995)). Modified plasmid vectors have been adapted specifically for direct injection (Hartikka et al., Hum. Gene Ther. 7(10):1205-17 (1996)).

Thus, a wide variety of gene transfer/gene therapy vectors and constructs are known in the art. These vectors are readily adapted for use in the methods of the present invention. By the appropriate manipulation using recombinant DNA/molecular biology techniques to insert an operatively linked Raf or Ras encoding nucleic acid segment (either active or inactive) into the selected expression/delivery vector, many equivalent vectors for the practice of the present invention can be generated.

F. Methods For Modulation of Angiogenesis

In one aspect, the present invention provides for a method for the modulation of angiogenesis in a tissue associated with a disease process or condition, and thereby affect events in the tissue which depend upon angiogenesis. Generally, the method comprises administering to the tissue, associated with, or suffering from a disease process or condition, an angiogenesis-modulating amount of a composition comprising a Raf protein or a nucleic acid vector expressing active or inactive Raf.

A further method comprises administering to the tissue, associated with a disease process or condition, an angiogenesis-modulating amount of a composition comprising a Ras protein or a nucleic acid vector expressing active or inactive Ras. Another method aspect comprises administering to the tissue associated with a disease process or condition, an angiogenesis-modulating amount of a Raf and Ras protein or one or more nucleic acid vector expressing active or inactive Raf and Ras.

Any of a variety of tissues, or organs comprised of organized tissues, can support angiogenesis in disease conditions including skin, muscle, gut, connective tissue, brain tissue, nerve cells, joints, bones and the like tissue in which blood vessels can invade upon angiogenic stimuli.

The patient to be treated according to the present invention in its many embodiments is a human patient, although the invention is effective with respect to all mammals. In this context, a “patient” is a human patient as well as a vetrinary patient, a mammal of any mammalian species in which treatment of tissue associated with diseases involving angiogenesis is desirable, particularly agricultural and domestic mammalian species.

Thus, the method embodying the present invention comprises administering to a patient a therapeutically effective amount of a physiologically tolerable composition containing a Raf and/or Ras protein or nucleic acid vector for expressing a Raf and/or Ras protein.

The dosage ranges for the administration of a Raf or Ras protein depend upon the form of the protein, and its potency, as described further herein, and are amounts large enough to produce the desired effect in which angiogenesis and the disease symptoms mediated by angiogenesis are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount of Raf or Ras protein, or nucleic acid encoding for (active or inactive) Raf or Ras protein, sufficient to produce a measurable modulation of angiogenesis in the tissue being treated, i.e., an angiogenesis-modulating amount. Modulation of angiogenesis can be measured or monitored in vitro by CAM assay as described herein, examination of tumor tissues, or by other methods known to one skilled in the art.

The Raf or Ras protein or nucleic acid vector expressing such protein can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, compositions of the invention can be administered intravenously, intraperitoneally, intramuscularly, subcutaneously, intracavity, transdermally, and can be delivered by peristaltic means, if desired.

The therapeutic compositions containing a Raf or Ras protein or nucleic acid vector expressing the Raf or Ras protein can be conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required physiologically acceptable diluent; i.e., carrier, or vehicle.

In one preferred embodiment the active material is administered in a single dosage intravenously. Localized administration can be accomplished by direct injection or by taking advantage of anatomically isolated compartments, isolating the microcirculation of target organ systems, reperfusion in a circulating system, or catheter based temporary occlusion of target regions of vasculature associated with diseased tissues.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgement of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

1. Inhibition of Angiogenesis

There are a variety of diseases in which inhibition of angiogenesis is important, referred to as angiogenic diseases, including but not limited to, inflammatory disorders such as immune and non-immune inflammation, chronic articular rheumatism and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth.

Thus, methods which inhibit angiogenesis in a tissue associated with a disease condition ameliorates symptoms of the disease and, depending upon the disease, can contribute to cure of the disease. In one embodiment, the invention contemplates inhibition of angiogenesis, per se, in a tissue associated with a disease condition. The extent of angiogenesis in a tissue, and therefore the extent of inhibition achieved by the present methods, can be evaluated by a variety of methods.

Thus, in one embodiment, a tissue to be treated is an inflamed tissue and the angiogenesis to be inhibited is inflamed tissue angiogenesis where there is neovascularization of inflamed tissue. This particular method includes inhibition of angiogenesis in arthritic tissues, such as in a patient with chronic articular rheumatism, in immune or non-immune inflamed tissues, in psoriatic tissue, and the like.

In another embodiment, a tissue to be treated is a retinal tissue of a patient suffering from a retinal disease such as diabetic retinopathy, macular degeneration or neovascular glaucoma and the angiogenesis to be inhibited is retinal tissue angiogenesis where there is neovascularization of retinal tissue.

In an additional embodiment, a tissue to be treated is a tumor tissue of a patient with a solid tumor, a metastases, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include lung, pancreas, breast, colon, laryngeal, ovarian, and the like tissues. Inhibition of tumor tissue angiogenesis is a particularly preferred embodiment because of the important role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor.

Stated in other words, the present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because (1) their formation requires vascularization of a primary tumor so that the metastatic cancer cells can exit the primary tumor and (2) their establishment in a secondary site requires neovascularization to support growth of the metastases.

In a yet further embodiment, the invention contemplates the practice of the method in conjunction with other therapies such as conventional chemotherapy directed against solid tumors and for control of establishment of metastases. The administration of angiogenesis inhibitor is typically conducted during or after chemotherapy, although it is preferably to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

Insofar as the present methods apply to inhibition of tumor neovascularization, the methods can also apply to inhibition of tumor tissue growth, to inhibition of tumor metastases formation, and to regression of established tumors.

Restenosis is a process of smooth muscle cell (SMC) migration and proliferation into the tissue at the site of percutaneous transluminal coronary angioplasty which hampers the success of angioplasty. The migration and proliferation of SMC's during restenosis can be considered a process of angiogenesis which is inhibited by the present methods. Therefore, the invention also contemplates inhibition of restenosis by inhibiting angiogenesis according to the present methods in a patient following angioplasty procedures. For inhibition of restenosis, the inactivated tyrosine kinase is typically administered after the angioplasty procedure because the coronary vessel wall is at risk of restenosis, typically for from about 2 to about 28 days, and more typically for about the first 14 days following the procedure.

The present method for inhibiting angiogenesis in a tissue associated with a disease condition, and therefore for also practicing the methods for treatment of angiogenesis-related diseases, comprises contacting a tissue in which angiogenesis is occurring, or is at risk for occurring, with a therapeutically effective amount of a composition comprising an inactivated Raf protein or vector expressing the protein. Inhibition of angiogenesis and tumor regression occurs as early as 7 days after the initial contacting with the therapeutic composition. Additional or prolonged exposure to inactive Raf or Ras protein is preferable for 7 days to 6 weeks, preferably about 14 to 28 days. Shorter periods of exposure can be useful where the modulating effects are detectable earlier, however administration and subsequent exposure for at least 12 hours is preferred.

2. Potentiation of Angiogenesis

In cases where it is desirable to promote or potentiate angiogenesis, administration of an active Raf or Ras protein to the tissue is useful. The routes and timing of administration are comparable to the methods described hereinabove for inhibition.

G. Therapeutic Compositions

The present invention contemplates therapeutic compositions useful for practicing the therapeutic methods described herein. Therapeutic compositions of the present invention contain a physiologically tolerable carrier together with a Raf or Ras protein or vector capable of expressing a Raf or Ras protein as described herein, dissolved or dispersed therein as an active ingredient. In a preferred embodiment, the therapeutic composition is not immunogenic when administered to a mammal or human patient for therapeutic purposes.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

A therapeutic composition contains an angiogenesis-modulating amount of a Raf or Ras protein of the present invention, or sufficient recombinant DNA expression vector to express an effective amount of Raf or Ras protein, typically formulated to contain an amount of at least 0.1 weight percent of Raf or Ras protein per weight of total therapeutic composition. A weight percent is a ratio by weight of Raf protein to total composition. Thus, for example, 0.1 weight percent is 0.1 grams of Raf or Ras protein per 100 grams of total composition. For DNA expression vectors, the amount administered depends on the properties of the expression vector, the tissue to be treated, and the like considerations. The suitable amount administered can be measured by amount of vector, or amount of expressed protein that is expected.

H. Article of Manufacture

The invention also contemplates an article of manufacture which is a labeled container for providing a Raf or Ras protein of the invention. An article of manufacture comprises packaging material and a pharmaceutical agent contained within the packaging material.

The pharmaceutical agent in an article of manufacture is any of the compositions of the present invention suitable for providing a Raf or Ras protein and formulated into a pharmaceutically acceptable form as described herein according to the disclosed indications. Thus, the composition can comprise a Raf and/or Ras protein or a DNA molecule which is capable of expressing a Raf and/or Ras protein. The article of manufacture contains an amount of pharmaceutical agent sufficient for use in treating a condition indicated herein, either in unit or multiple dosages.

The packaging material comprises a label which indicates the use of the pharmaceutical agent contained therein, e.g., for treating conditions assisted by the inhibition or potentiation of angiogenesis, and the like conditions disclosed herein. The label can further include instructions for use and related information as may be required for marketing. The packaging material can include container(s) for storage of the pharmaceutical agent.

As used herein, the term packaging material refers to a material such as glass, plastic, paper, foil, and the like capable of holding within fixed means a pharmaceutical agent. Thus, for example, the packaging material can be plastic or glass vials, laminated envelopes and the like containers used to contain a pharmaceutical composition including the pharmaceutical agent.

In preferred embodiments, the packaging material includes a label that is a tangible expression describing the contents of the article of manufacture and the use of the pharmaceutical agent contained therein.

### EXAMPLES

The following examples relating to this invention are illustrative and should not, of course, be construed as specifically limiting the invention. Moreover, such variations of the invention, now known or later developed, which would be within the purview of one skilled in the art are to be considered to fall within the scope of the present invention hereinafter claimed.

1. Preparation of c-Raf Expression Constructs

For preparing the expression constructs useful in modulating angiogenesis by the methods of the present invention, c-Raf cDNA is manipulated and inserted into an expression construct/vector.

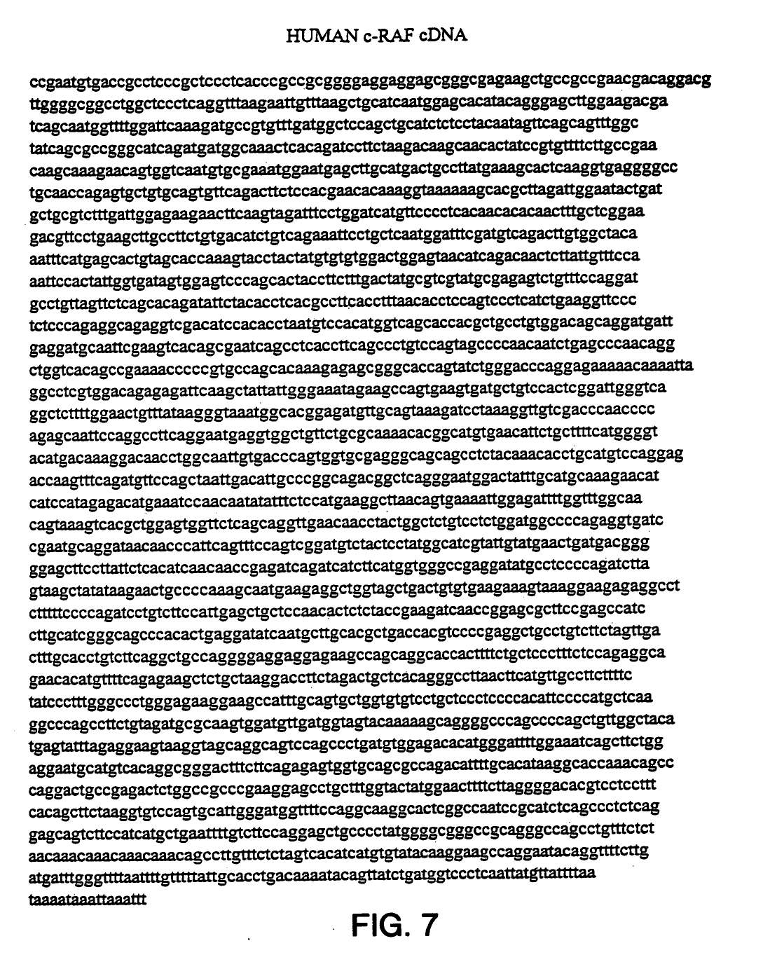

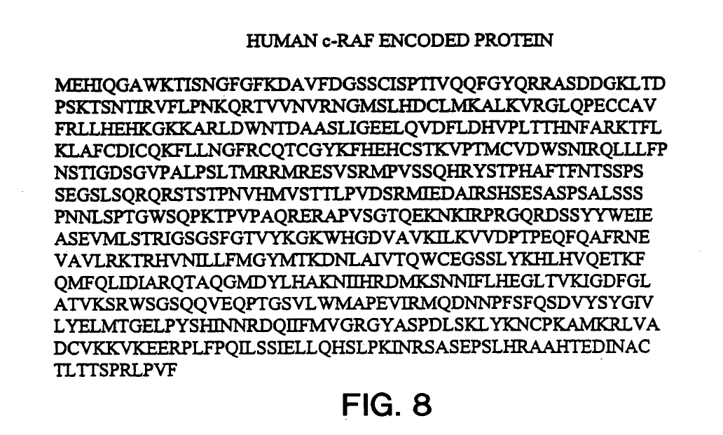

The cDNA sequence encoding for wild-type (i.e., endogenous) human c-Raf is depicted in the nucleic acid sequence shown in FIG. 7 (SEQ ID NO.: 1, nucleotides 130 . . . 2076) with the encoded translated amino acid residue sequence for the Raf protein depicted in FIG. 8 (SEQ ID NO.: 2).

The present invention describes two categories of c-Raf function to modulate angiogenesis. As previously discussed, one category contains Raf molecules that increase angiogenesis and, thus, are considered to be active proteins. Wild-type Raf along with various mutations are shown in the present invention to induce angiogenesis.

One preferred mutation of wild type c-Raf which functions in this context with respect to its ability to induce blood vessel growth and therefore increase tumor weight in vivo is the Raf mutant construct in which only the amino acid residues 306-648 of Raf (Raf 306-648) are expressed. This construct lacks the entire regulatory kinase domain and is therefore referred to as a constitutively active Raf protein.

Mutations in Raf have also been shown to have the opposite modulatory effect on angiogenesis, inhibiting angiogenesis instead of stimulating it. Such mutations are referred to as inactive Raf mutations. Proteins having mutations that confer this inhibitory activity are also referred to as dominant negative Raf proteins in that they inhibit neovascularization, including that resulting from endogenous activity of Raf as well as enhanced Raf activity resulting from growth factor stimulation. Thus, certain mutations of wild type c-Raf of the present invention can also function as a dominant negative with respect to their ability to block blood vessel growth, and for example, therefore decrease tumor weight in vivo.

An exemplary inhibitory Raf construct is the Raf mutation in which the lysine amino acid residue 375 is mutated into any other amino acid, preferably a methionine (i.e., Raf K375M). This point mutation in the kinase domain prevents ATP binding and also blocks kinase-dependent Raf functions related to vascular cell and tumor cell signaling and proliferation. Another inhibitory Raf mutant would comprise amino acid residues 1-305 in the form of a truncated Raf protein (i.e., Raf 1-305), which lacks the kinase domain.

With respect to the point mutations, any mutation resulting in the desired inhibitory or stimulatory activity is contemplated for use in this invention. Fusion protein constructs combining the desired Raf protein (mutation or fragment thereof) with expressed amino acid tags, antigenic epitopes, fluorescent protein, or other such protein or peptides are also contemplated, so long as the desired modulating effect of the Raf protein is intact.

To produce the desired c-Raf mutations in the cDNA, standard site-directed mutagenesis procedures familiar to one of ordinary skill in the art were utilized. PCR primers designed to incorporate the desired mutations were also designed with restriction sites to facilitate subsequent cloning steps. Entire segments of Raf encoding nucleic acid sequences are deleted from the nucleic acid constructs through PCR amplification techniques based on the known cDNA sequences of chicken, human and the like homologs of Raf and subsequent formation of new constructs.

Specifically, the wild-type Raf cDNA sequence shown in FIG. 7 was modified in several ways to construct Raf mutants to demonstrate the principles of the present invention. These mutants were inserted into the retrovirus expression system described herein.

A first mutant Raf, designated Raf K375M, was constructed in wild-type human Raf in which lysine at amino acid residue position 375 was substituted by a methionine. Raf K375M is an “inactive” Raf protein as defined herein.

A second mutant Raf, designated Raf 306-648, was constructed in wild-type human Raf in which the amino terminal portion was deleted, leaving the truncated carboxy terminal residues 306-648. Raf 306-648 is an “active” Raf protein as defined herein.

A third mutant Raf, designated Raf 1-305, is constructed in wild type human Raf in which the carboxy terminal portion was deleted, leaving the truncated amino terminal residues 1-305. Raf 1-305 is an “inactive” Raf protein as defined herein.

Alternative expression vectors for use in the expressing the Raf or Ras proteins of the present invention also include adenoviral vectors as described in U.S. Pat. No. 4,797,368, No. 5,173,414, No. 5,436,146, No. 5,589,377, and No. 5,670,488. Alternative methods for the delivery of the Raf or Ras modulatory proteins include delivery of the Raf or Ras cDNA with a non-viral vector system as described in U.S. Pat. No. 5,675,954 and delivery of the cDNA itself as naked DNA as described in U.S. Pat. No. 5,589,466. Delivery of constructs of this invention is also not limited to topical application of a viral vector, viral vector preparations are also injected intravenously for systemic delivery into the vascular bed, or can be injected subcutaneously, intratissue, and the like. These vectors are also targetable to sites of increased neovascularization by localized injection of a tumor, as an example.

In vitro expressed proteins are also contemplated for delivery thereof following expression and purification of the selected Raf or Ras protein by methods useful for delivery of proteins or polypeptides. One such method includes liposome delivery systems, such as described in U.S. Pat. No. 4,356,167, No. 5,580,575, No. 5,542,935 and No. 5,643,599. Other vector and protein delivery systems are well known to those of ordinary skill in the art for use in the expression and/or delivery of the Raf or Ras proteins of the present invention.

2. Human Tumor Model

To demonstrate the efficacy of the present invention, human tumor cells were implanted subcutaneously onto the flank of athymic mice, and allowed to grow to about 100 mm³. In this xenograft model, the murine endothelial cells in the tissue surrounding the implant form vasculature that grow into the growing human tumor in response to the normal angiogenic signals, and the tumor becomes vascularized. Thus, the microvessels are formed by murine endothelial cells, whereas the tumor tissue itself comprises human cells.

3. Retrovirus Delivery Vector Infects Mouse Lineage Cells, Not Human Tumor Cells

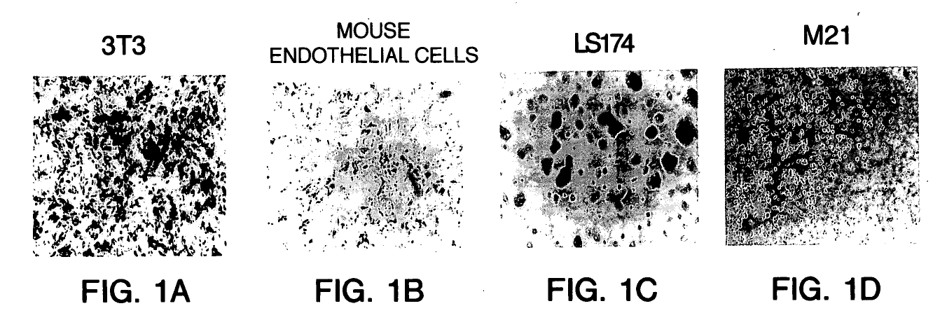

The retrovirus expression vector system of Clonetech was used to construct ecotrophic retrovirus which contain the constructs of Raf described herein. To demonstrate the tissue specificity of the infecting retrovirus, a retrovirus expression vector construct which expresses b-galactosidase was packaged using ecotrophic packaging cells as described in the legend to FIG. 1.

Mouse 3T3, mouse endothelial cells, human epithelial adenocarcinoma LS174 cells and human melanoma M21 cells were cultured in vitro, and were each exposed to the ecotrophically packaged retrovirus. Only the murine cells express detectable b-galactosidase, indicating that only murine cells are infected by ecotrophically packaged retrovirus in this expression system.

4. Inactive Raf Kinase Disrupts Raf Kinase Activity In Vitro

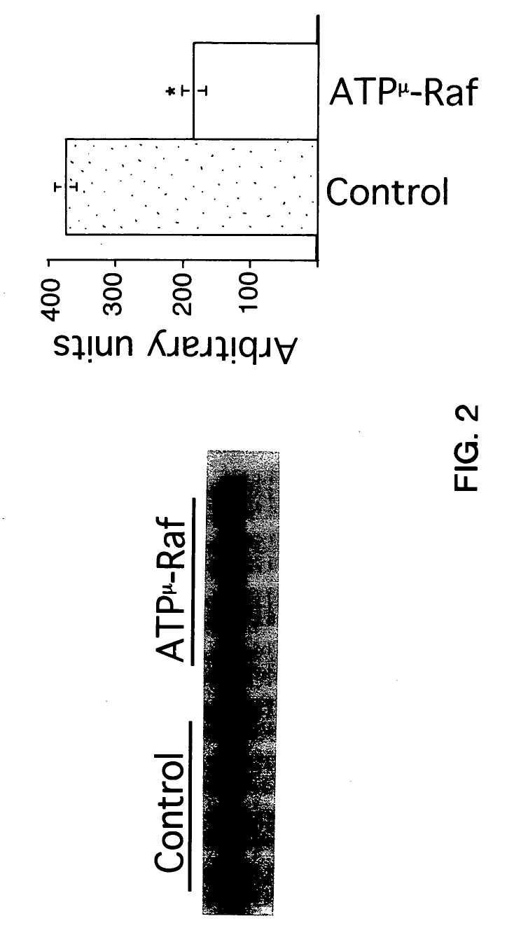

To demonstrate the cellular effects of inactive Raf kinase, an in vitro model using mouse endothelial cells induced by bFGF was used. The normal induction of Raf activity by bFGF administration to mouse endothelial cells was blocked when those cells were first infected by a retroviral construct which expressed the inactive Raf K375M kinase construct as described in the legend to FIG. 2. The data in FIG. 2 shows that the amount of Raf kinase activity is substantially reduced when cells are first infected by the vector which expresses an inactive Raf kinase.

5. Inactive Raf Kinase Disrupts Angiogenesis In Vivo

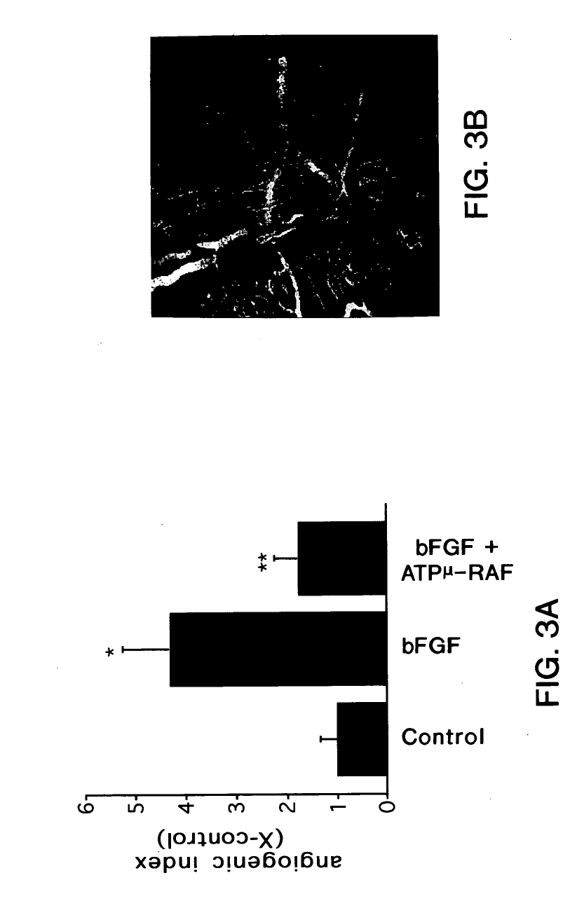

Using an in vivo murine subcutaneous model for angiogenesis, the effects of inactive Raf kinase were studied. To that end, angiogenesis was induced in a mouse by injection of bFGF either with or without cells expressing retrovirus that produces the inactive Raf K375M kinase protein as described in the legend to FIG. 3. As shown in FIG. 3, the presence of inactive Raf kinase substantially reduced the angiogenic index.

6. Active Raf Kinase Induces Angiogenesis In Vivo

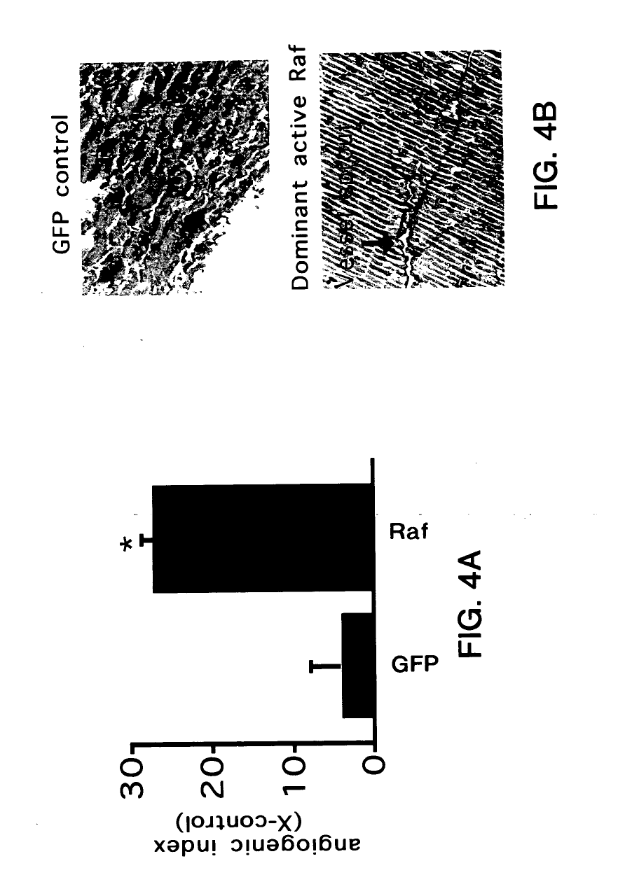

Using the murine subcutaneous model for angiogenesis, the effects of active Raf kinase were studied. To that end, angiogenesis was induced by injection of cells expressing retrovirus that produced the active Raf 306-648 kinase as described in the legend to FIG. 4. As shown in FIG. 4, mutationally active Raf kinase induces angiogenesis in vivo.

7. Inactive Raf Kinase Induces Apoptosis

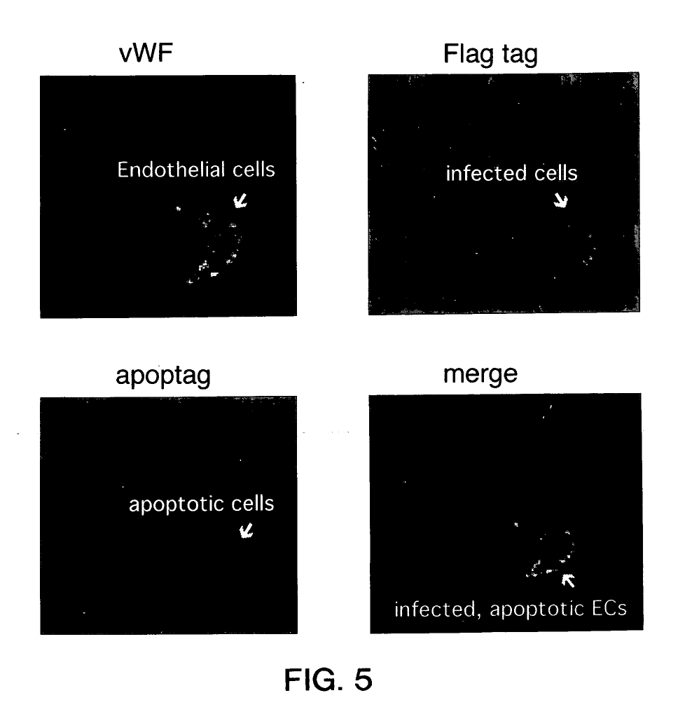

Using the mouse xenograft model described above, the in vivo effects of inactive Raf were studied. To that end, the model was established as described in the legend to FIG. 5 by injection of 1.5 million human adenocarcinoma LS174 cells. Following establishment of a tumor mass of about 100 mm³, retrovirus expressing the inactive Raf K375M kinase were injected into the tumor mass, and immunohistochemistry was performed on sections of the tumor mass after 48 hours. The results shown in FIG. 5 (Flag tag) indicate that the retrovirus infection was endothelial specific, and further shows via the vWF stain that the endothelial cells colocalized to the retrovirus infection. The merge of the staining data shows that the endothelial cells, the virus infection and the occurrence of apoptosis all colocalized, indicating that the virus delivery of the inactive Raf protein is endothelial specific and that inactive Raf induces apoptosis.

8. Inactive Raf Kinase Induces Tumor Regression

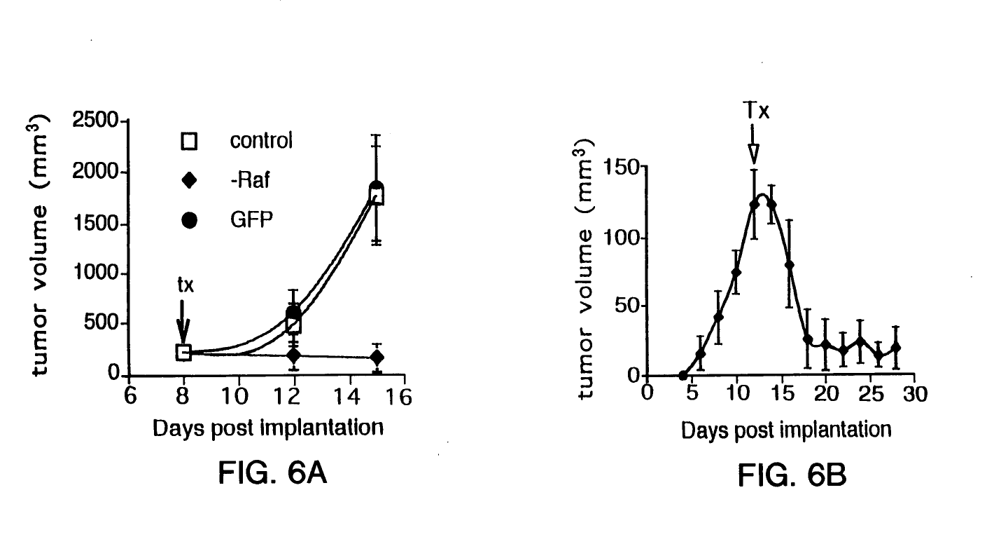

Using the mouse xenograft model described above, the in vivo effects of inactive Raf on tumor regression were studied. To that end, the model was established as described in the legend to FIG. 6, and the inactive Raf K375M kinase was provided as virus supernate or virus-expressing cells as indicated. The established tumor was seen to rapidly regress upon introduction of inactive Raf kinase.

9. Angiogenesis is Dependent on Activation of the Ras-Raf-MEK-ERK Pathway

To determine the interaction of growth factor receptor and integrin receptor ligation and activation on the activation of the mitogen-activated protein kinase (MAPK)/extracellular signal-regulated kinase (ERK) cascade that is involved in modulating angiogenesis, the following studies in Examples 9-11 were performed. Activation of the MAPK cascade by integrin-mediated cell adhesion has been investigated by a number of laboratories as reviewed by Aplin et al., Pharmacol. Rev., 50:197-263 (1998). The hierarchical ERK cascade originates at the cell membrane with receptors for mitogens and growth factors which recruits the small guanosine triphosphate (GTPase) Ras which then activates Raf, a protein kinase, by binding to Raf and recruiting it to the membrane, where it is activated in a yet undetermined mechanism. Activated Raf then phosphorylates and activates MEK (MAPK/ERK kinase). MEK, then, phosphorylates and activates ERK1 and ERK2 which then translocate to the nucleus and transactivate transcription factors to effect growth, differentiation or mitosis through altered gene expression. (See, Tibbles et al., Cell Mol. Life Sci., 55:1230-1254 (1999)).

The upstream regulation of Ras in activation of Raf that is mediated by growth factor and/or integrin signaling is the subject of current studies but the mechanisms of signaling are still not completely understood. (See, Stewart et al., J. Biol. Chem., 275:8854-8862 (2000); Howe et al., J. Biol. Chem., 273:27268-27274 (1998)). However, and more importantly, the activation of the Ras-Raf-MEK-ERK cascade through cell membrane receptor signaling resulting in modulation of angiogenesis has not been described before the present invention.

A. Ras is Induced by Exposure to bFGF

Therefore, to first assess whether angiogenesis was dependent on the Ras-Raf-MEK-ERK pathway, Ras activity was measured in chick chorioallantoic membrane (CAM) lysates exposed to bFGF as determined by a Ras pulldown assay.

Angiogenesis can be induced on the CAM after normal embryonic angiogenesis has resulted in the formation of mature blood vessels. Angiogenesis has been shown to be induced in response to specific cytokines or tumor fragments as described by Leibovich et al., Nature, 329:630 (1987) and Ausprunk et al., Am. J. Pathol., 79:597 (1975). CAMs were prepared from chick embryos for subsequent induction of angiogenesis and inhibition thereof. Ten day old chick embryos were obtained from McIntyre Poultry (Lakeside, Calif.) and incubated at 37° C. with 60% humidity. A small hole was made through the shell at the end of the egg directly over the air sac with the use of a small crafts drill (Dremel, Division of Emerson Electric Co. Racine Wis.). A second hole was drilled on the broad side of the egg in a region devoid of embryonic blood vessels determined previously by candling the egg. Negative pressure was applied to the original hole, which resulted in the CAM (chorioallantoic membrane) pulling away from the shell membrane and creating a false air sac over the CAM. A 1.0 centimeter (cm)×1.0 cm square window was cut through the shell over the dropped CAM with the use of a small model grinding wheel (Dremel). The small window allowed direct access to the underlying CAM.

The resultant CAM preparation was then used at 10 days of embryogenesis where angiogenesis has subsided. The latter preparation was, thus, used in this invention for inducing renewed angiogenesis in response to cytokine treatment or tumor contact, where necessary, as described below.

1) Angiogenesis Induced by Growth Factors

Angiogenesis has been shown to be induced by cytokines or growth factors. Angiogenesis was induced by placing a 5 millimeter (mm)×5 mm Whatman filter disk (Whatman Filter paper No. 1) saturated with Hanks Balanced Salt Solution (HBSS, GIBCO, Grand Island, N.Y.) or HBSS containing recombinant basic fibroblast growth factor (bFGF) or vascular endothelial cell growth factor (VEGF) (Genzyme, Cambridge, Mass.) on the CAM of either a 9 or 10 day chick embryo in a region devoid of blood vessels and the windows were latter sealed with tape. Other growth factors are also effective at inducing blood vessel growth. For assays where inhibition of angiogenesis is evaluated with intravenous injections of antagonists, such as LM609 monoclonal antibody, angiogenesis is first induced with bFGF or VEGF in fibroblast growth medium, and then inhibitors are administered as described in Example 10. Angiogenesis was monitored by photomicroscopy after 72 hours.

CAMs from 10-day old chick embryos were stimulated topically with filter disks saturated with either PBS or 30 nanograms (ng) of bFGF. After 5 minutes, CAM tissue was resected, homogenized in lysis buffer, and Ras activity was then determined by its capacity to be precipitated by a GST fusion peptide encoding the Ras binding domain of Raf. Because only active Ras binds Raf, a recombinant protein was generated consisting of the Ras binding domain of Raf conjugated to glutathione-S-transferase (GST). In turn GST was conjugated to sepharose beads enabling the precipitation of active Ras from a tissue lysate.

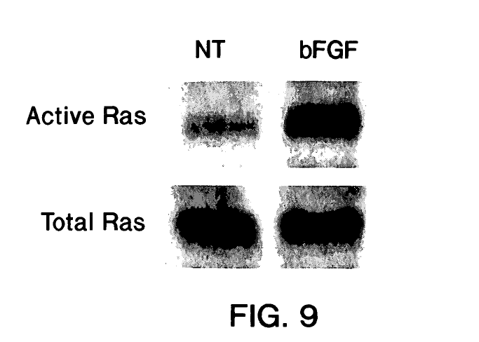

The results are shown in FIG. 9 where Ras activity was elevated in CAM lysates exposed to bFGF as determined by a Ras pulldown assay. Thus, Ras is induced with exposure to bFGF in the CAM. The role of Ras in the formation of angiogenic blood vessels in the CAM is further assessed as described in Example 10.

B. Ras is Necessary for Angiogenesis

To then determine whether angiogenesis was dependent on the activation of Ras in the CAM preparation, the CAM was exposed to RCAS retroviral preparations for expression of a dominant negative Ras mutant, S17N Ras, in combination with bFGF activation of Ras as described below. This mutant has been shown to bind GDP with preferential affinity over GTP, thereby providing the mutant to inhibit endogenous Ras activation by sequestering Ras-GEFs. Thus, use of the mutant in the CAM angiogenesis model provides a method to assess the role of Ras in angiogenesis.

The S17N Ras mutant is created from the wild-type human Ras (wt H-Ras) sequence by standard site directed mutagenesis procedures as previously described substituting the encoding triplet for a serine (S) residue at position 17 with a codon for encoding an asparagine (N). Such mutants have been described by others, for example, by Stewart et al., J. Biol. Chem., 275:8854-8862 (2000).

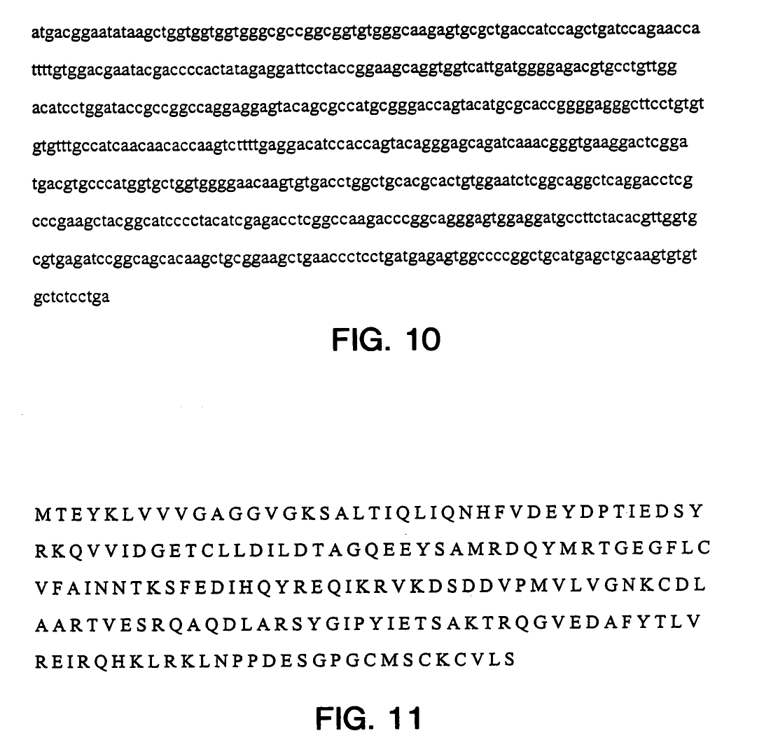

To prepare the retroviral construct of the dominant negative expression construct, such mutagenesis was performed on the wt H-Ras, where the nucleic acid sequence encoding it is shown in FIG. 10 (SEQ ID NO.: 3). FIG. 11 (SEQ ID NO.: 4) depicts the amino acid residue sequence encoded by the cDNA nucleotide sequence of wild-type human Ras (wt H-Ras) shown in FIG. 10. To produce the desired mutations in the wt H-Ras cDNA to make S17N Ras as well as those described below, standard site-directed mutagenesis procedures familiar to one of ordinary skill in the art were utilized. PCR primers designed to incorporate the desired mutations were also designed with restriction sites to facilitate subsequent cloning steps. Entire segments of Ras encoding nucleic acid sequences can be deleted from the nucleic acid constructs through PCR amplification techniques based on the known cDNA sequences of chicken, human and the like homologs of Ras and subsequent formation of new constructs. All mutant constructs constructed by PCR were also sequenced by PCR to confirm predicted DNA sequence of clones.

The resultant mutated Ras sequence was then prepared as an retroviral expression vector construct as described herein. One preferred expression construct for use in the present invention is the RCAS(A) construct. This expression vector is based on a series of replication competent avian sarcoma viruses with an enhanced Bryan polymerase (BP) for improved titre, and is specific for the A type envelope glycoprotein expressed on normal avian cells (Reviewed in Methods in Cell Biology, 52:179-214 (1997); see also, Hughes et al., J. Virol. 61:3004-3012 (1987); Fekete & Cepko, Mol. Cellular Biol. 13:2604-2613 (1993); Itoh et al., Development 122:291-300 (1996); and Stott et al., BioTechniques 24:660-666 (1998)). The complete sequence of RCAS(A), referred to herein as RCAS, is known to one of ordinary skill in the art and available on databases.

Five micrograms (ug) of RCAS constructs prepared were then transfected into the chicken immortalized fibroblast line, DF-1 (gift of Doug Foster, U. of Minn.). This cell line as well as primary chick embryo fibroblasts were capable of producing virus, however the DF-1 cell line produced higher titres. Viral supernatants were collected from subconfluent DF-1 producer cell lines in serum free CLM media [composition: F-10 media base supplemented with DMSO, folic acid, glutamic acid, and MEM vitamin solution]. Thirty-five ml of viral supernatant were concentrated by ultracentrifugation at 4° C. for 2 hours at 22,000 rpm. These concentrated viral pellets were resuspended in 1/100 the original volume in serum-free CLM media, aliquoted and stored at −80° C. The titre was assessed by serial dilution of a control viral vector having a nucleotide sequence encoding green fluorescent protein (GFP), referred to as RCAS-GFP, infection on primary chick embryo fibroblasts that were incubated for 48-72 hours. The titres of viral stock that were obtained following concentration routinely exceeded 108 I.u./ml.

For the CAM assay using the viral stocks, cortisone acetate soaked Whatman filter disks 6 mm in diameter were prepared in 3 mg/ml cortisone acetate for 30 minutes in 95% ethanol. The disks were dried in a laminar flow hood and then soaked on 20 μl of viral stock per disk for 10 minutes. These disks were applied to the CAM of a 10 day chick embryos and sealed with cellophane tape and incubated at 37° C. for 18-24 hr. Then either mock PBS or growth factors were added at a concentration of 5 μg/ml to the CAM in a 15 microliters (ul) volume of the appropriate virus stock as an additional boost of virus to the CAM tissue. After 72 hours, the CAMs were harvested and examined for changes in the angiogenic index as determined by double blind counting of the number of branch points in the CAM underlying the disk. For kinase assays, the tissue underlying the disk was harvested in RIPA, homogenized with a motorized grinder and Raf determined as previously described in Example 4. For immunofluorescence studies, CAM tissue underlying the disks were frozen in OCT, a cryopreservative, sectioned at 4 um, fixed in acetone for 1 minute, incubated in 3% normal goat serum for 1 hour, followed by an incubation in primary rabbit antibody as described previously (Eliceiri et al., J. Cell Biol., 140:1255-1263 (1998), washed in PBS and detected with a fluorescent secondary antibody.

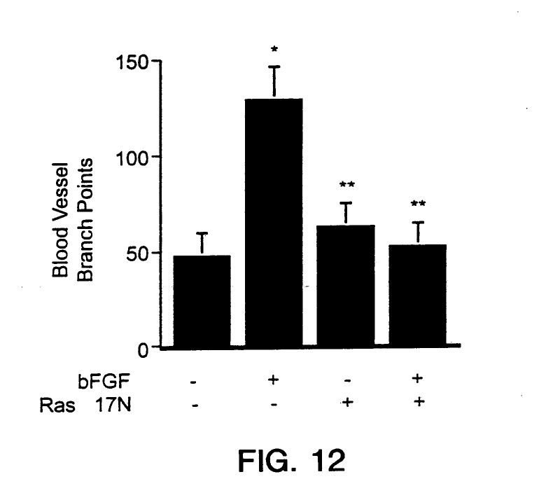

The results, shown in FIG. 12, graphically reveal that infection with mutant null Ras, S17N, blocked growth factor-induced angiogenesis in the CAM, but had no effect on CAMs that were not exposed to bFGF to induce angiogenesis. Therefore, Ras is necessary for bFGF-induced angiogenesis.

C. Ras Signaling Through the Raf-MEK-ERK Pathway is a Crucial Regulator of Angiogenesis

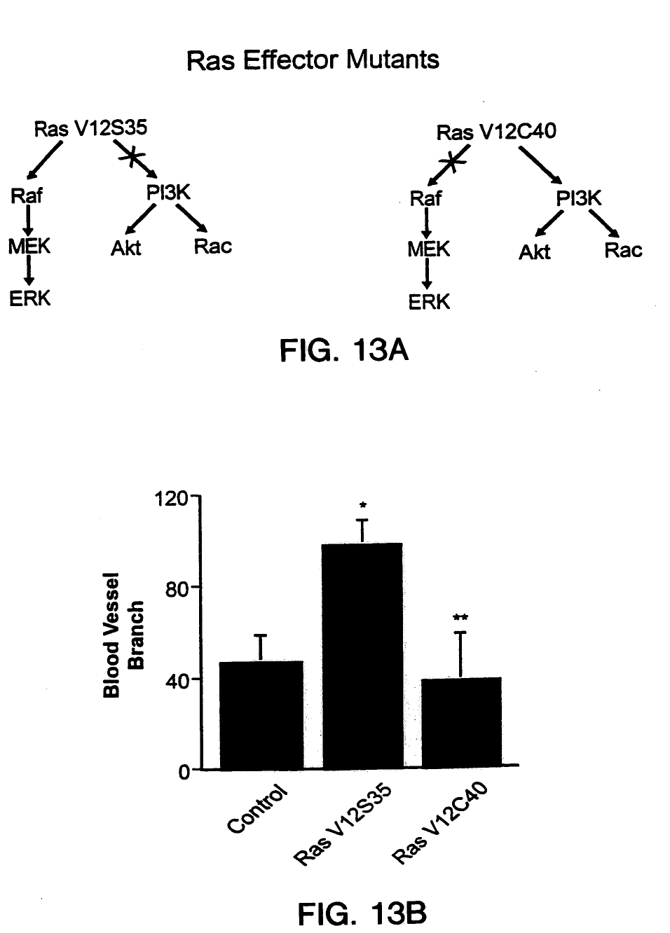

To further assess the role of Ras in the Raf-MEK-ERK pathway in modulating angiogenesis, additional H-Ras mutant proteins were used in the CAM preparation as described above, the results of which are shown below and in FIG. 13. In this context, the present invention describes two categories of Ras function that can modulate angiogenesis. As previously discussed for Raf proteins, one category contains Ras molecules that increase angiogenesis and, thus, are considered to be active proteins. Wild-type Ras along with various mutations are shown in the present invention to induce angiogenesis.

One preferred mutation of wild type H-Ras which functions in this context with respect to its ability to induce blood vessel growth and therefore increase tumor weight in vivo is the Ras G12V, also referred to as V12, mutant having a point mutation at amino acid (aa) residue position 12 changing glycine (G) to valine (V). This mutant Ras is constitutively active.

Another H-Ras mutant protein that is described for the present invention as a constitutive angiogenesis activator is Ras V12S35, where the glycine at position 12 was changed to valine (V) and the threonine (T) at position 35 was changed to a serine (S), both mutations resulting in Ras V12S35. This mutated H-Ras protein has been shown to only selectively activate the Raf-MEK-ERK pathway as shown in FIG. 13A.

A H-Ras negative regulator of angiogenesis is Ras V12C40 mutant, where the glycine at position 12 was changed to valine (V) as in Ras V12S35 but the other mutation was at position 40 where a tyrosine residue (Y) was changed to a cysteine (C), both mutations, thus, resulting in Ras V12C40. This mutant H-Ras is known to selectively activate the P1-3 kinase (P13K as shown in FIG. 13A) pathway that activates Akt and Rac. Thus, Ras V12C40 does not function in the Raf-MEK-ERK pathway and does not stimulate angiogenesis but rather would inhibit it. Proteins having mutation that confer inhibitory activity on angiogenesis are also referred to as dominant negative Ras proteins in that they inhibit neovascularization, including that resulting from endogenous activity of Ras as well as enhanced Ras activity resulting from growth factor stimulation. Thus, certain mutations of wild type H-Ras of the present invention can also function as a dominant negative with respect to their ability to block blood vessel growth, and for example, therefore decrease tumor weight in vivo. The three H-Ras constructs and mutant proteins have been previously described by Joneson et al., Science, 271:810-812 (1996).

With respect to the point mutations, any mutation resulting in the desired inhibitory or stimulatory activity is contemplated for use in this invention. Fusion protein constructs combining the desired Ras (or Raf proteins as shown in the Examples below) (mutation or fragment thereof) with expressed amino acid tags, antigenic epitopes, fluorescent protein, or other such protein or peptides are also contemplated, so long as the desired modulating effect of the Ras protein is intact.

To evaluate the roles of the additional Ras mutant proteins in signaling pathway activation of angiogenesis, the respective retroviral expression constructs were prepared as described above. Fifteen ul of high titer RCAS (A) virus encoding the Raf-MEK-ERK activating Ras construct, Ras V12S35, or the P13 kinase activating Ras construct, Ras V12C40, were topically applied to filter disks in a 10-day old CAM preparation and results assessed as described above for the effect of the mutant Ras proteins on angiogenesis with respect to the selective activation of signaling pathways.

FIGS. 13A and 13B illustrate schematically and graphically respectively that infection with a mutant Ras construct, Ras V12S35, which selectively activates the Ras-Raf-MEK-ERK pathway, induced angiogenesis, whereas a mutant construct, Ras V12C40, which selectively activates the PI3K pathways did not. Thus, these results confirm that Ras V12S35 protein is a angiogenesis stimulator and that Ras-mediated activation of angiogenesis occurs through activation of the Raf-MEK-ERK pathway and not via the P13K pathway utilized by the H-Ras mutant V12C40.

D. The MEK Component of the MEK-ERK Pathway is Required for Either Ras or Ras-Independent Raf Induced Angiogenesis

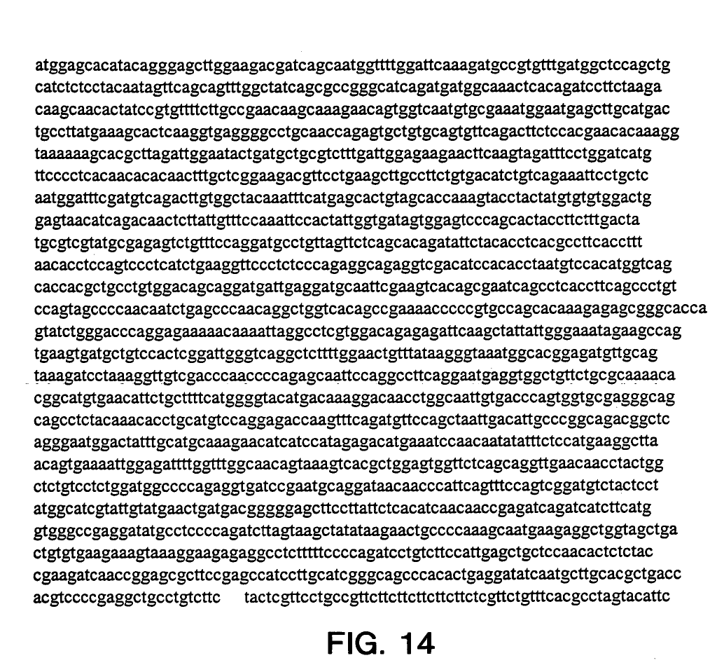

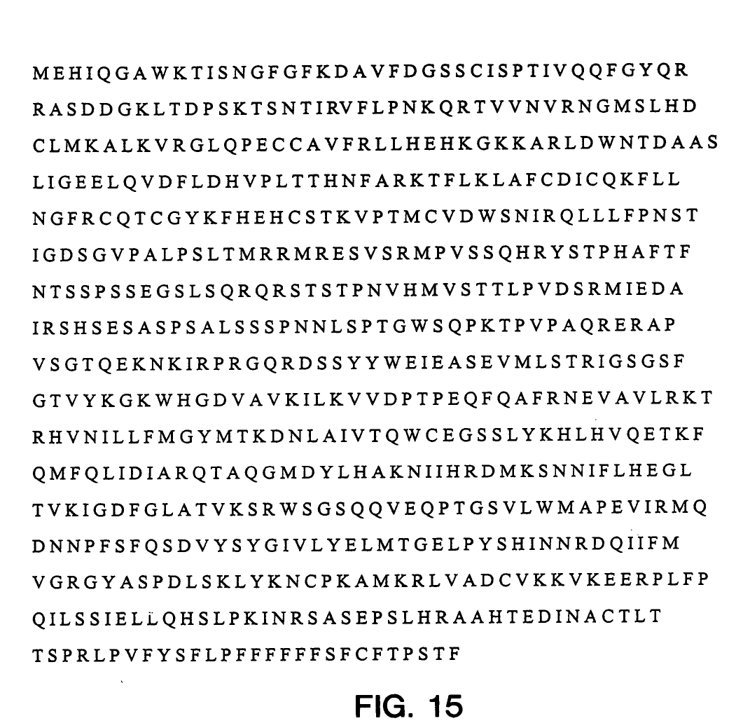

To further assess the separate roles of Ras and Raf in the Raf-MEK-ERK pathway in modulating angiogenesis, a Raf mutant protein, referred to as Raf-Caax, that is targeted to the plasma membrane that is known to be constitutively and enzymatically active in the absence of Ras binding was used in the CAM preparations as described herein in conjunction with a known inhibitor of MEK activation, PD98059. FIG. 14 depicts the nucleotide sequence encoding the fusion protein Raf-caax, where the nucleotide sequence encoding the carboxy terminus of human Raf (wt H-Raf) is fused with a nucleotide sequence of encoding a 20 amino acid residue sequence of the K-ras membrane localization domain (SEQ ID NO.: 6). FIG. 15 (SEQ ID NO.: 7) depicts the amino acid residue sequence of Raf-caax, the fusion protein generated from the fusion nucleotide sequence shown in FIG. 14. The fusion protein has been described by Leevers et al., Nature, 369:411-414 (1994) and Stokoe et al., Science, 264:1463-1467 (1994).

For assessing the Ras-independent Raf-induced angiogenesis along with angiogenesis induced by Raf, the MEK inhibitor, PD98059, was used in CAM preparations as described above. Virus encoding the activating Ras construct, Ras V12 (Ras G12V), prepared as described in Example 9C and the activating Raf construct, Raf-caax, were topically applied to filter disks as described in Example 9B. After 24 hours, one (1) nanomole of the MEK inhibitor, PD98059, was added to the disk. The CAMs were then evaluated as described in Example 9B and in FIG. 12. Data plotted is the mean±SE of 20 embryos.

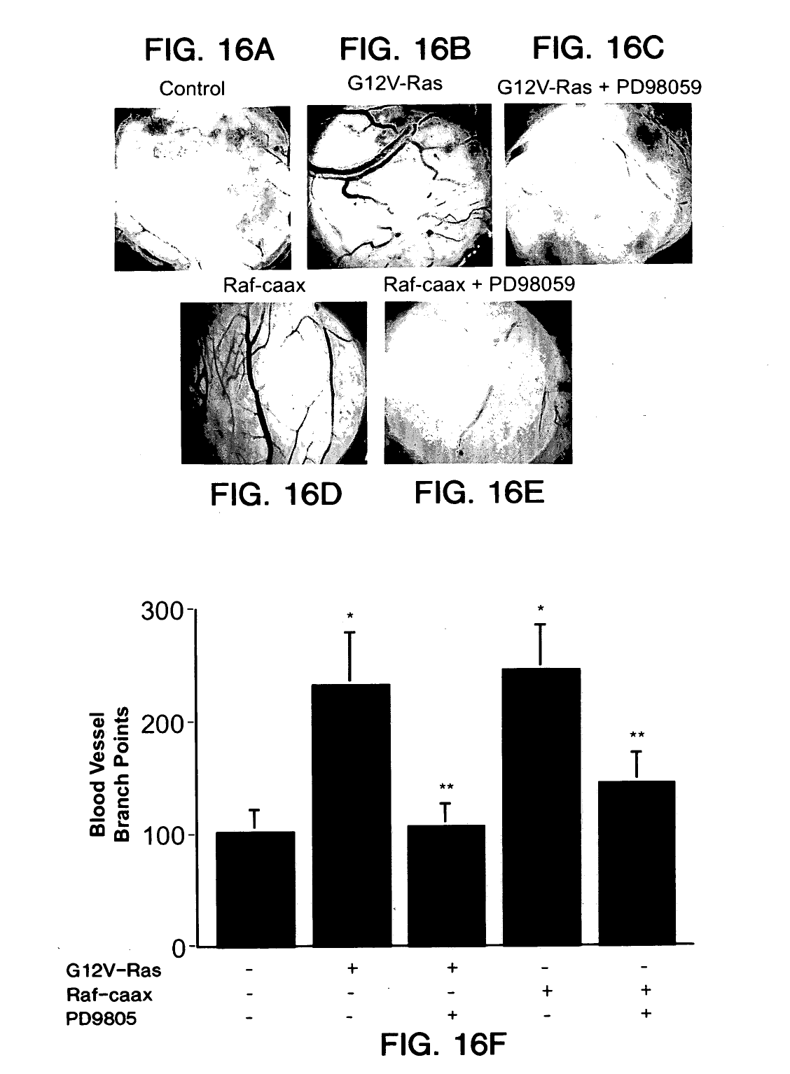

FIGS. 16A-16E and FIG. 16F, respectively, pictorially and graphically illustrate that the MEK inhibitor, PD98059, blocked angiogenesis (FIGS. 16C and 16E) induced by either mutant active Ras (FIG. 16B) or Raf (FIG. 16D). Thus, both Ras and Raf induce angiogenesis through the MEK-ERK pathway. The plotted data graphically depicts the results of the photographs of the individual treated CAMs.

10. Angiogenesis Induced by Raf, but not Ras, is Refractory to Inhibition by Integrin Blockade

To determine how integrin signaling activates the Ras-Raf-MEK-ERK pathway resulting in angiogenesis, CAM assays with mutant active Ras and Raf constructs were performed in the presence of αᵥβ₃ integrin-blocking antibodies. CAMs from 10-day old chick embryos were stimulated as described in FIGS. 9 and 12 with filter disks saturated with either PBS (control), bFGF, the RCAS(A) retroviral constructs G12V-Ras or Raf-caax. LM4609, a monoclonal antibody to integrin αᵥβ₃ was intravenously delivered after 24 hours and angiogenesis was assessed by vessel branch point analysis after 72 hours. Representative CAMs are shown in the inset. Data is the mean±SE of 20 embryos.

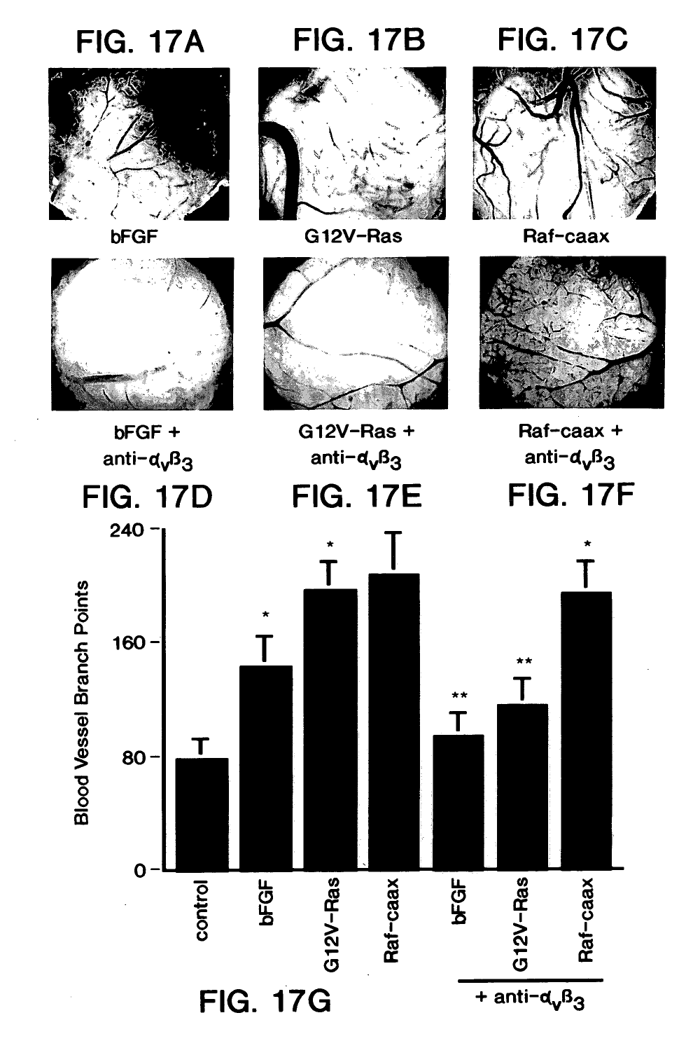

FIGS. 17A-17F and FIG. 17G, respectively, pictorially and graphically illustrate that angiogenesis induced by Raf, but not Ras, was refractory to inhibition by integrin blockade. Infection with both mutant active Ras and Raf constructs induced pronounced angiogenesis as shown respectively in FIGS. 17B and 17C, but only Ras-induced angiogenesis was inhibited by αᵥβ₃ integrin-blocking antibodies as shown in FIG. 17E. Since the Raf construct used in the assay is Ras-independent, the lack of integrin inhibition of Raf-induced angiogenesis indicates that integrin signaling occurs at or before Ras-mediated activation of Raf. The plotted data graphically depicts the results of the photographs of the individual treated CAMs.

11. Regulation of the Ras-Raf-MEK-ERK Pathway by Focal Adhesion Kinase

To determine the role of growth factor receptor activation of the Ras-Raf-MEK-ERK angiogenesis pathway, CAM angiogenesis assays were performed as described above with either Ras V12 or Raf-caax expressed proteins in the presence of a mutant null focal adhesion kinase, referred to as FRNK, which is an inactive focal adhesion kinase.

RCAS(A) viruses encoding Ras V12 or Raf-caax, prepared as described above, were topically applied as described in Example 9B (FIG. 12) along with RCAS(B) virus encoding FAK-related-null-kinase (FRNK) to the CAM filter disk. Data is the mean±SE of 20 embryos.

The results are shown in FIGS. 18A-18D and 18E. FIGS. 18A-18D pictorially illustrate that co-infection of CAMs with a mutant null focal adhesion kinase, FRNK, blocked Ras, but not Raf-induced angiogenesis, as indicated by a paucity of blood vessels in FIG. 18B as compared to untreated Ras (FIG. 18A), untreated Raf (FIG. 18C) and FRNK-treated Raf (FIG. 18D). The plotted data graphically depicts the results of the photographs of the individual treated CAMs.

The data in the CAM assay was confirmed in the murine subcutaneous angiogenesis model, prepared as previously described. Angiogenesis was induced by injecting 250 ul of ice-cold, growth factor-reduced matrigel containing either 400 ng/ml bFGF or Moloney retrovirus expressing packaging cells expressing the described gene, subcutaneously in the mouse flank. FRNK retrovirus was added to matrigel as high titer virus packaged with the vsv.g coat protein. Five days later, endothelial-specific FITC-conjugated Bandeiriea Simplifica B5 lectin was injected via the tail vein and allowed to circulate. Angiogenesis was then quantitated by removing, extracting, and assaying the angiogenic tissue for fluorescent content.

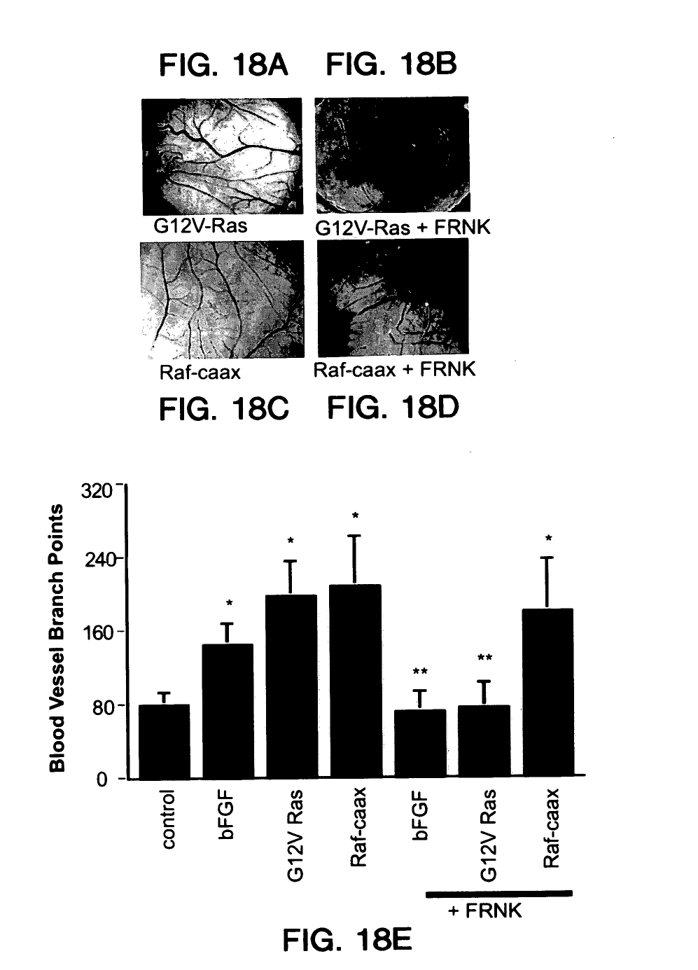

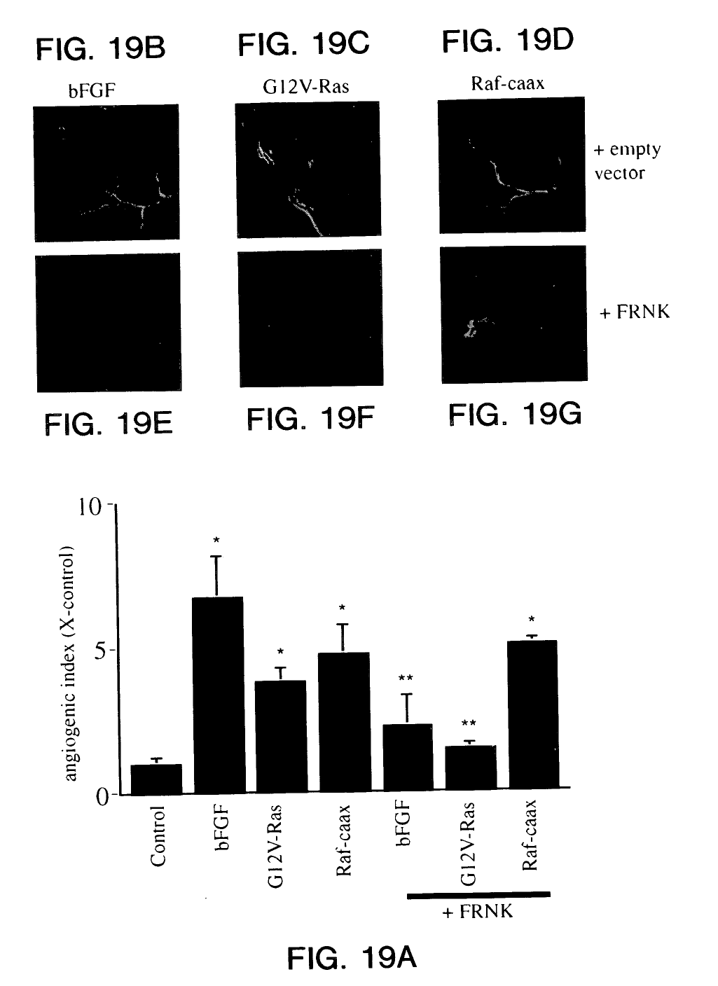

FIGS. 19A and 19B-19G, respectively, graphically and pictorially, illustrate that FRNK blocked bFGF and Ras-, but not Raf, -induced angiogenesis in a murine subcutaneous angiogenesis model.

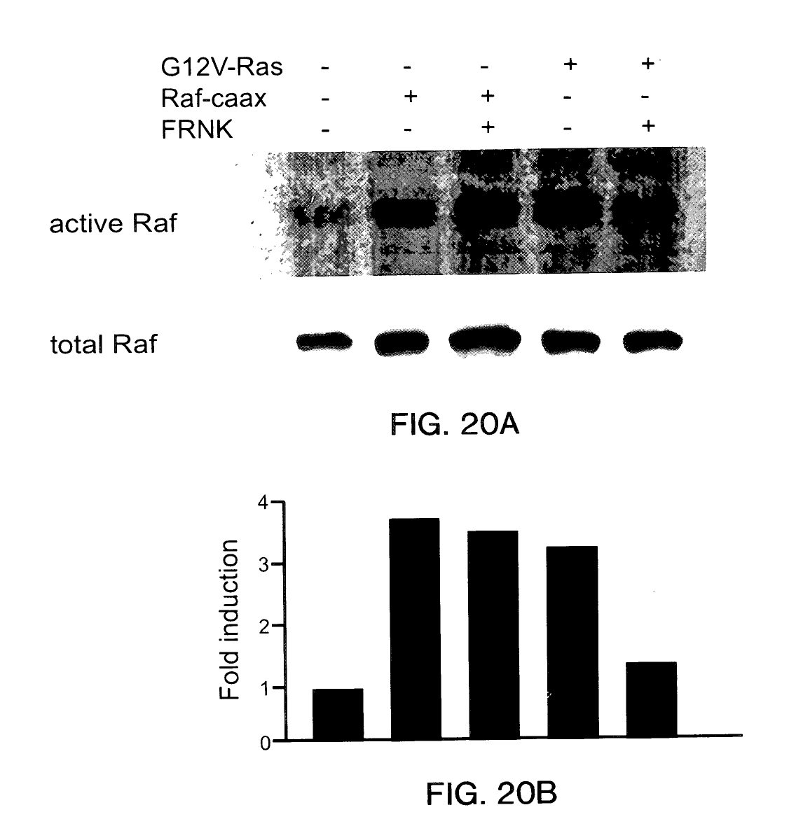

To verify the level at which kinase activation occurs in the Ras-Raf-MEK-ERK pathway, CAMS were co-infected with a retrovirus expressing FRNK, the mutant null focal adhesion kinase, with either Ras G12V or Raf-caax. CAMs were treated as described in FIG. 18 with the exception that after 24 hours the angiogenic tissue was resected, solubilized, Raf immunoprecipitated, and Raf activity assessed by its capacity to phosphorylate kinase-dead MEK. FIGS. 20A and 20B illustrate that co-infection of CAMs with a mutant null focal adhesion kinase, FRNK, blocked Ras-induced activation of Raf. FIG. 20A shows the immunoprecipated active versus total Raf proteins assayed under each of the combinations above the results. FIG. 20B graphically plots the results of the active Raf determinations under those conditions. Thus, FRNK does not directly inhibit the activity of Raf but rather inhibits the activation of Raf by Ras.

12. Discussion

The above studies indicates that Raf kinase is necessary and sufficient for angiogenesis in vivo. Further, targeting of mutationally inactive Raf kinase to growing blood vessels induces local endothelial apoptosis. The same targeting also suppresses angiogenesis which results in suppression and even regression of pre-existing human tumors.

The retroviral delivery of a gene encoding mutationally inactive forms of Raf kinase (Raf K375M) demonstrated a substantial impact on tumor angiogenesis in vivo. Importantly, the retroviral vector used specifically infects proliferating cells of murine lineage. Therefore, only the vascular compartment of human tumor xenografts was infected (FIGS. 1 and 4). Delivery of inactive Raf K375M kinase was found to suppress growth factor-induced Raf kinase activity in vitro and block growth factor-induced angiogenesis in vivo (FIGS. 2 & 3). In contrast, retroviral delivery of a mutationally active form of Raf kinase (Raf 306-648) was sufficient to induce angiogenesis in vivo (FIG. 4). Furthermore, the delivery of virus expressing inactive Raf kinase to the tumor in mice was found to induce apoptosis in a endothelial-specific manner (FIG. 5). Finally, animals inoculated with human tumors and then treated with the virus expressing inactive Raf experienced a rapid tumor regression which was maintained throughout the time-course of the experiment (FIG. 6). Therefore, Raf kinase is both sufficient and necessary for angiogenesis and targeting this kinase can suppress angiogenesis and obviate angiogenesis-dependent disease.

As a result of the foregoing angiogenesis assays in mouse and chicken as described in Examples 9-11, depicted in FIGS. 9, 12, 13, and 16-20, the present invention provides angiogenesis activator proteins in Raf-caax, Ras G12V Ras, and Ras V12S35 and angiogenesis inhibitor proteins in Ras S17N and Ras V12C40. Furthermore, the studies provide the basis for understanding the Ras-mediated activation of Raf in the Ras-Raf-MEK-ERK pathway identifying that Ras is necessary for activation of Raf but integrin-mediated signaling interacts at of before Raf activation but not downstream thereof.

While the foregoing written specification is sufficient to enable one skilled in the art to practice the invention, various modifications of the invention in addition to those shown and described herein will become apparent to those skilled in the art from the foregoing description and fall within the scope of the appended claims.

We claim:

1. An article of manufacture comprising packaging material and a pharmaceutical composition contained within said packaging material, wherein said pharmaceutical composition is capable of modulating angiogenesis in a tissue associated with a disease condition, wherein said packaging material comprises a label which indicates that said pharmaceutical composition can be used for treating disease conditions by modulating angiogenesis, and wherein said pharmaceutical composition comprises an oligonucleotide having a nucleotide sequence capable of expressing a Raf protein.

2. The article of manufacture of claim 1 wherein said Raf protein is an active Raf protein.

3. The article of manufacture of claim 2 wherein said active Raf protein is wild-type Raf.

4. The article of manufacture of claim 3 wherein said active Raf protein is a fusion protein.

5. The article of manufacture of claim 4 wherein said active Raf fusion protein is Raf-caax.

6. The article of manufacture of claim 1 wherein said Raf protein is an inactive Raf protein.

7. The article of manufacture of claim 6 wherein said inactive Raf protein has a mutation at residue 375 such that the amino acid at position 375 is not lysine.

8. The article of manufacture of claim 1 wherein said pharmaceutical composition further comprises a liposome.

9. The article of manufacture of claim 1 wherein said pharmaceutical composition comprises a viral expression vector capable of expressing said nucleotide sequence.

10. The article of manufacture of claim 1 wherein said pharmaceutical composition comprises an non-viral expression vector capable of expressing said nucleotide sequence.

11. A method for modulating angiogenesis in a tissue associated with a disease condition comprising administering to said tissue an angiogenesis modulating amount of a pharmaceutical composition comprising a nucleotide sequence capable of expressing a Raf protein.

12. The method of claim 11 wherein said Raf protein is an active Raf protein and said modulating potentiates angiogenesis.

13. The method of claim 12 wherein said active Raf protein is wild-type Raf.

14. The method of claim 13 wherein said active Raf protein is a fusion protein.

15. The method of claim 14 wherein said active Raf fusion protein is Raf-caax.

16. The method of claim 12 wherein said tissue has abnormal circulation.

17. The method of claim 11 wherein said Raf protein is an inactive Raf protein and said modulating inhibits angiogenesis.

18. The method of claim 17 wherein said inactive Raf protein has a mutation at residue 375 such that the amino acid at position 375 is not lysine.

19. The method of claim 17 wherein said tissue is inflamed and said condition is arthritis or rheumatoid arthritis.

20. The method of claim 17 wherein said tissue is a solid tumor or solid tumor metastasis.

21. The method of claim 20 wherein said administering is conducted in conjunction with chemotherapy.

22. The method of claim 17 wherein said tissue is retinal tissue and said condition is retinopathy, diabetic retinopathy or macular degeneration.

23. The method of claim 17 wherein said tissue is at the site of coronary angioplasty and said tissue is at risk for restenosis.

24. The method of claim 11 wherein said administering comprises a single dose intravenously.

25. A pharmaceutical composition for stimulating angiogenesis in a target mammalian tissue comprising a gene transfer vector containing a nucleic acid, said nucleic acid having a nucleic acid segment encoding for a Raf protein and a pharmaceutically acceptable carrier or excipient.

26. A method for modulating angiogenesis in a tissue associated with a disease condition comprising administering to said tissue an angiogenesis modulating amount of a pharmaceutical composition comprising a nucleotide sequence capable of expressing Raf protein, and a nucleotide sequence capable of expressing Ras protein.

27. A method of claim 26 wherein said modulation is an inhibition of angiogenesis, and at least one of said Raf and Ras proteins is inactive.

28. A method of claim 26 wherein said modulation is an stimulation of angiogenesis, and at least one of said Raf and Ras proteins is active.


SEQUENCE LISTING (7 sequences)

SEQ ID NO: 1 - DNA, 2977 bp, Homo sapiens
  CDS (130)...(2073)
ccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccacgctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcaggagcagtcttccatcatgctgaattttgtcttccaggagctgcccctatggggcgggccgcagggccagcctgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaattt

SEQ ID NO: 2 - protein, 648 aa, Homo sapiens [sequence omitted]

SEQ ID NO: 3 - DNA, 570 bp, Homo sapiens
  CDS (1)...(567)
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctga

SEQ ID NO: 4 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 5 - DNA, 6453 bp, Homo sapiens
  prim_transcript (1664)...(3744)
  intron (1775)...(2041)
  intron (2534)...(3230)
ggatcccagcctttccccagcccgtagccccgggacctccgcggtgggcggcgccgcgctgccggcgcagggagggcctctggtgcaccggcaccgctgagtcgggttctctcgccggcctgttcccgggagagcccggggccctgctcggagatgccgccccgggcccccagacaccggctccctggccttcctcgagcaaccccgagctcggctccggtctccagccaagcccaaccccgagaggccgcggccctactggctccgcctcccgcgttgctcccggaagccccgcccgaccgcggctcctgacagacgggccgctcagccaaccggggtggggcggggcccgatggcgcgcagccaatggtaggccgcgcctggcagacggacgggcgcggggcggggcgtgcgcaggcccgcccgagtctccgccgcccgtgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtgagtgcgggcagggatcggccgggccgcgcgccctcctcgcccccaggcggcagcaatacgcgcggcgcgggccgggggcgcggggccggcgggcgtaagcggcggcggcggcggcgggtgggtggggccgggcggggcccgcgggcacaggtgagcgggcgtcgggggctgcggcgggcgggggccccttcctccctggggcctgcgggaatccgggccccacccgtggcctcgcgctgggcacggtccccacgccggcgtacccgggagcctcgggcccggcgccctcacacccgggggcgtctgggaggaggcggccgcggccacggcacgcccgggcacccccgattcagcatcacaggtcgcggaccaggccgggggcctcagccccagtgccttttccctctccgggtctcccgcgccgcttctcggccccttcctgtcgctcagtccctgcttcccaggagctcctctgtcttctccagctttctgtggctgaaagatgcccccggttccccgccgggggtgcggggcgctgcccgggtctgccctcccctcggcggcgcctagtacgcagtaggcgctcagcaaatacttgtcggaggcaccagcgccgcggggcctgcaggctggcactagcctgcccgggcacgccgtggcgcgctccgccgtggccagacctgttctggaggacggtaacctcagccctcgggcgcctccctttagcctttctgccgacccagcagcttctaatttgggtgcgtggttgagagcgctcagctgtcagccctgcctttgagggctgggtcccttttcccatcactgggtcattaagagcaagtgggggcgaggcgacagccctcccgcacgctgggttgcagctgcacaggtaggcacgctgcagtccttgctgcctggcgttggggcccagggaccgctgtgggtttgcccttcagatggccctgccagcagctgccctgtggggcctggggctgggcctgggcctggctgagcagggccctccttggcaggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggtgagcctagcgccgccgtccaggtgccagcagctgctgcgggcgagcccaggacacagccaggatagggctggctgcagcccctggtcccctgcatggtgctgtggccctgtctcctgcttcctctagaggaggggagtccctcgtctcagcaccccaggagaggagggggcatgaggggcatgagaggtaccagggagaggctggctgtgtgaactccccccacggaaggtcctgagggggtccctgagccctgtcctcctgcaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacaggtgaaccccgtgaggctggcccgggagcccacgccgcacaggtggggccaggccggctgcgtccaggcaggggcctcctgtcctctctgcgcatgtcctggatgccgctgcgcctgcagcccccgtagccagctctcgctttccacctctcagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcaggtgaggcagctctccaccccacagctagccagggacccgccccgccccgccccagccagggagcagcactcactgaccctctcccttgacacagggcagccgctctggctctagctccagctccgggaccctctgggaccccccgggacccatgtgacccagcggcccctcgcactgtaggtctcccgggacggcagggcagtgagggaggcgagggccggggtctgggctcacgccctgcagtcctgggccgacacagctccggggaaggcggaggtccttggggagagctgccctgagccaggccggagcggtgaccctggggcccggcccctcttgtccccagagtgtcccacgggcacctgttggttctgagtcttagtggggctactggggacacgggccgtagctgagtcgagagctgggtgcagggtggtcaaaccctggccagacctggagttcaggagggccccgggccaccctgacctttgaggggctgctgtagcatgatgcgggtggccctgggcacttcgagatggccagagtccagcttcccgtgtgtgtggtgggcctggggaagtggctggtggagtcgggagcttcgggccaggcaaggcttgatcccacagcagggagcccctcacccaggcaggcggccacaggccggtccctcctgatcccatccctcctttcccagggagtggaggatgccttctacacgttggtgcgtgagatccggcagcacaagctgcggaagctgaaccctcctgatgagagtggccccggctgcatgagctgcaagtgtgtgctctcctgacgcaggtgagggggactcccagggcggccgccacgcccaccggatgaccccggctccccgcccctgccggtctcctggcctgcggtcagcagcctcccttgtgccccgcccagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgatcttggttttcggctgagggtgggacacggtgcgcgtgtggcctggcatgaggtatgtcggaacctcaggcctgtccagccctgggctctccatagcctttgggagggggaggttgggagaggccggtcaggggtctgggctgtggtgctctctcctcccgcctgccccagtgtccacggcttctggcagagagctctggacaagcaggcagatcataaggacagagagcttactgtgcttctaccaactaggagggcgtcctggtcctccagagggaggtggtttcaggggttggggatctgtgccggtggctctggtctctgctgggagccttcttggcggtgagaggcatcacctttcctgacttgctcccagcgtgaaatgcacctgccaagaatggcagacatagggaccccgcctcctgggccttcacatgcccagttttcttcggctctgtggcctgaagcggtctgtggaccttggaagtagggctccagcaccgactggcctcaggcctctgcctcattggtggtcgggtagcggccagtagggcgtgggagcctggccatccctgcctcctggagtggacgaggttggcagctggtccgtctgctcctgccccactctcccccgcccctgccctcaccctacccttgccccacgcctgcctcatggctggttgctcttggagcctggtagtgtcactggctcagccttgctgggtatacacaggctctgccacccactctgctccaaggggcttgccctgccttgggccaagttctaggtctggccacagccacagacagctcagtcccctgtgtggtcatcctggcttctgctgggggcccacagcgcccctggtgcccctcccctcccagggcccgggttgaggctgggccaggccctctgggacggggacttgtgccctgtcagggttccctatccctgaggttgggggagagctagcagggcatgccgctggctggccagggctgcagggacactcccccttttgtccagggaataccacactcgcccttctctccagcgaacaccacactcgcccttctctccaggggacgccacactcccccttctgtccaggggacgccacactcccccttctctccaggggacgccacactcgcccttctctccaggggacgccacactcgcccttctctccaggggacgccacactcgcccttctgtccaggggacgccacactcgcccttctctccaggggacgccacactcgcccttctctccaggggacgccacactcccccttctgtccaggggacgccacactcccccttctctccaggggacgccacactcccccttctctccaggggacgccacactcgcccttctctccaggggacgccacactcccccttctgtccaggggacgccacactcgcccttctctccaggggacgccacactcgcccttctctccaggggacgccacactcccccttctctccaggggacgccacactcccccttctctccaggggacgccacactcccccttctgtccaggggacgccacactcgcccttctctccaggggacgccacactcccccttctctccaggggacgccacactcccccttctctccaggggacgccacactcccccttctgtccaggggacgccacactcgcccttctctccaggggacgccacactcgcccttctctccaggggacgccacactcgcccttctctccaggggacgccacacttgcccttctgtccagggaatgccacactcccccttctccccagcagcctccgagtgaccagcttccccatcgatagacttcccgaggccaggagccctctagggctgccgggtgccaccctggctccttccacaccgtgctggtcactgcctgctgggggcgtcagatgcaggtgaccctgtgcaggaggtatctctggacctgcctcttggtcattacggggctgggcagggcctggtatcagggccccgctggggttgcagggctgggcctgtgctgtggtcctggggtgtccaggacagacgtggaggggtcagggcccagcacccctgctccatgctgaactgtgggaagcatccaggtccctgggtggcttcaacaggagttccagcacgggaaccactggacaacctggggtgtgtcctgatctggggacaggccagccacaccccgagtcctagggactccagagagcagcccactgccctgggctccacggaagccccctcatgccgctaggccttggcctcggggacagcccagctaggccagtgtgtggcaggaccaggcccccatgtgggagctgaccccttgggattctggagctgtgctgatgggcaggggagagccagctcctccccttgagggagggtcttgatgcctggggttacccgcagaggcctgggtgccgggacgctccccggtttggctgaaaggaaagcagatgtggtcagcttctccactgagcccatctggtcttcccggggctgggccccatagatctgggtccctgtgtggcccccctggtctgatgccgaggatacccctgcaaactgccaatcccagaggacaagactgggaagtccctgcagggagagcccatccccgcaccctgacccacaagagggactcctgctgcccaccaggcatccctccagggatcc

SEQ ID NO: 6 - DNA, 2004 bp, Artificial Sequence
  Description of Artificial Sequence fusion protein
atggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccacgctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctactcgttcctgccgttcttcttcttcttcttctcgttctgtttcacgcctagtacattc

SEQ ID NO: 7 - protein, 668 aa, Artificial Sequence
  Description of Artificial Sequence fusion protein
MEHIQGAWKTISNGFGFKDAVFDGSSCISPTIVQQFGYQRRASDDGKLTDPSKTSNTIRVFLPNKQRTVVNVRNGMSLHDCLMKALKVRGLQPECCAVFRLLHEHKGKKARLDWNTDAASLIGEELQVDFLDHVPLTTHNFARKTFLKLAFCDICQKFLLNGFRCQTCGYKFHEHCSTKVPTMCVDWSNIRQLLLFPNSTIGDSGVPALPSLTMRRMRESVSRMPVSSQHRYSTPHAFTFNTSSPSSEGSLSQRQRSTSTPNVHMVSTTLPVDSRMIEDAIRSHSESASPSALSSSPNNLSPTGWSQPKTPVPAQRERAPVSGTQEKNKIRPRGQRDSSYYWEIEASEVMLSTRIGSGSFGTVYKGKWHGDVAVKILKVVDPTPEQFQAFRNEVAVLRKTRHVNILLFMGYMTKDNLAIVTQWCEGSSLYKHLHVQETKFQMFQLIDIARQTAQGMDYLHAKNIIHRDMKSNNIFLHEGLTVKIGDFGLATVKSRWSGSQQVEQPTGSVLWMAPEVIRMQDNNPFSFQSDVYSYGIVLYELMTGELPYSHINNRDQIIFMVGRGYASPDLSKLYKNCPKAMKRLVADCVKKVKEERPLFPQILSSIELLQHSLPKINRSASEPSLHRAAHTEDINACTLTTSPRLPVFYSFLPFFFFFFSFCFTPSTF